(19) United States
(12) Patent Application Publication
(10) Pub. No.: US 2021/0225495 A1
(43) Pub. Date: Jul. 22, 2021

(54) SYSTEMS AND METHODS FOR ADAPTING A UI BASED PLATFORM ON PATIENT MEDICAL DATA

(71) Applicant: Nunetz, Inc., Wilmington, DE (US)

(72) Inventor: Tal RUSAK, Loudonville, NY (US)

(73) Assignee: Nunetz, Inc., Wilmington, DE (US)

(21) Appl. No.: 17/055,581

(22) PCT Filed: May 15, 2019
(86) PCT No.: PCT/IB2019/054030

§ 371 Date: Nov. 15, 2020

Related U.S. Application Data
(60) Provisional application No. 62/671,540, filed on May 15, 2018.

(51) Int. Cl. G16H 40/20; G16H 10/60; G16H 40/67; G16H 50/20; G16H 50/70; G16H 15/00; G16H 30/20; G16H 30/40

(52) U.S. Cl. CPC G16H 40/20; G16H 10/60; G16H 40/67; G16H 50/20; G06F 3/167; G16H 15/00; G16H 30/20; G16H 30/40; G16H 50/70

(57) ABSTRACT

There is provided a method of adapting a user interface (UI) for presenting medical data of a target patient, comprising: monitoring an interaction journey of a healthcare provider with at least one medical device that performs at least one member of the group consisting of: storing data of a target patient, monitoring the target patient, presenting medical data of the target patient, and treating the target patient, monitoring at least one patient parameter of the target patient, feeding the interaction journey and the at least one patient parameter into a model trained according to computed at least correlation between interaction journeys of at least one of sample healthcare providers with respective medical devices and at least one patient parameter of at least one sample patient, and outputting an adaptation to the UI by the model.

### RELATED APPLICATION

This application claims the benefit of priority under 35 USC § 119(e) of U.S. Provisional Patent Application No. 62/671,540 filed May 15, 2018, the contents of which are incorporated herein by reference in their entirety.

### BACKGROUND

The present invention, in some embodiments thereof, relates to the technical field of analysis of medical data of a patient and, more specifically, but not exclusively, to adaptation of a user interface (UI) based on an analysis of medical data of a patient.

A large amount of medical data is available for each patient, for example, data collected by sensors (e.g., pulse oximeter, blood pressure monitor), data in the electronic medical record, and medical images. Moreover, aggregated data collected from multiple subjects, which may help in planning treatment of the target patient, is also available in large quantities, for example, in the form of clinical guidelines and/or published research reports.

Traditionally, physicians manually review the patient data, and may manually review relevant published research and/or guidelines for determining the current clinical state of the patient and planning treatment. Such manual review tends to be difficult, tedious, time consuming, and prone to error due to missed data.

### SUMMARY

According to a first aspect, a method of adapting a user interface (UI) for presenting medical data of a target patient, comprises: monitoring an interaction journey of a healthcare provider with at least one medical device that performs at least one member of the group consisting of: storing data of a target patient, monitoring the target patient, presenting medical data of the target patient, and treating the target patient, monitoring at least one patient parameter of the target patient, feeding the interaction journey and the at least one patient parameter into a model trained according to at least one computed correlation between interaction journeys of at least one of sample healthcare providers with respective medical devices and at least one patient parameter of at least one sample patient, and outputting an adaptation to the UI by the model.

According to a second aspect, a system for adapting a user interface (UI) for presenting medical data of a target patient, comprises: at least one hardware processor executing a code for: monitoring an interaction journey of a healthcare provider with at least one medical device that performs at least one member of the group consisting of: storing data of a target patient, monitoring the target patient, presenting medical data of the target patient, and treating the target patient, monitoring at least one patient parameter of the target patient, feeding the interaction journey and the at least one patient parameter into a model trained according to at least one computed correlation between interaction journeys of at least one of sample healthcare providers with respective medical devices and at least one patient parameter of at least one sample patient, and outputting an adaptation to the UI by the model.

According to a third aspect, a computer program product for adapting a user interface (UI) for presenting medical data of a target patient, comprises: a non-transitory memory storing thereon code for execution by at least one hardware process, the code including instructions for: monitoring an interaction journey of a healthcare provider with at least one medical device that performs at least one member of the group consisting of: storing data of a target patient, monitoring the target patient, presenting medical data of the target patient, and treating the target patient, monitoring at least one patient parameter of the target patient, feeding the interaction journey and the at least one patient parameter into a model trained according to at least one computed correlation between interaction journeys of at least one of sample healthcare providers with respective medical devices and at least one patient parameter of at least one sample patient, and outputting an adaptation to the UI by the model.

In a further implementation of the first, second, and third aspects, the at least one medical device is selected from the group consisting of: electronic health information system, electronic medical record, medical imaging system, medical image, patient monitor, anesthesiology monitor, physiological sensor or monitor, intracranial pressure sensor, cerebral perfusion pressure sensor, arterial line, respiration device, blood pressure sensor, temperature sensor, and pulse oximeter.

In a further implementation of the first, second, and third aspects, the interaction journey is computed based on at least one member of the group consisting of: analysis of images captured by a camera of the healthcare provider interacting with the respective medical device, analysis of interaction of the healthcare provider with a dedicated display of the respective medical device, analysis of interaction of the healthcare provider with a generic display of a computing device connected to the respective medical device, and analysis of interaction of the healthcare provider with buttons of the respective medical device, interface of the medical device outputting data indicative of the interaction, and a microphone capturing interactions of healthcare providers with each other, interactions of healthcare providers with patients, and/or dictated patient notes, notes of patients.

In a further implementation of the first, second, and third aspects, further comprising obtaining at least one of sound and images and data of an interaction of the healthcare provider with at least one of the patient and other healthcare providers, and feeding the at least one of sound and images and data of the interaction into the model, wherein the model is trained on at least one of sound and images and data of a plurality of interactions of other healthcare providers with other patients and/or with another set of healthcare providers.

In a further implementation of the first, second, and third aspects, the interaction is selected from the group of: referral to another healthcare provider.

In a further implementation of the first, second, and third aspects, the interaction journey is of a healthcare provider with a UI presented on a display.

In a further implementation of the first, second, and third aspects, further comprising creating an adapted UI according to the adaptation outputted by the model, and iterating the monitoring the interaction journey, the monitoring the at least one patient parameter, the feeding, the outputting and the adapting, wherein the monitoring comprises monitoring the interaction journey of the healthcare provider with the adapted UI presented on the display.

In a further implementation of the first, second, and third aspects, the at least one patient parameter is presented on the UI.

In a further implementation of the first, second, and third aspects, the interaction journey is based on at least one member of the group consisting of: touch interaction with a touch screen presenting the UI, pen capable of writing on the touch screen, dial or wheel placed on a screen presenting the UI, a keyboard, a port receiving data from another user interface device or a network, a microphone, a virtual reality (VR) device, an augmented reality (AR) device, and/or a camera capturing images of the interaction with the UI.

In a further implementation of the first, second, and third aspects, an interaction included in the interaction journey is selected from the group consisting of, zoom-in on a certain monitored patient parameter, selection of a certain monitored patient parameter for presentation in the UI, removal of a certain monitored patient parameter from the UI, relative positioning between two or more monitored patient parameters in the UI, marking a certain monitored patient parameter with an indication of importance, entering data, entering a diagnosis, entering orders for treatment, receiving patient parameter from a medical device and/or system, observing a change in a patient parameter.

In a further implementation of the first, second, and third aspects, the interaction journey is computed based on at least one member selected from the group consisting of a camera capturing images of the healthcare provider treating the patient, a camera capturing images of healthcare provider actions when not directly treating the patient, a camera capturing images of healthcare provider washing hands, a camera capturing images of patient events including cough, seizure, sneeze, and/or fall, a microphone recording sounds captured during the patient events, a microphone recording sound captured during activities taking place in proximity to the patient, a microphone recording sound captured of the healthcare provider, and interactions of the healthcare provider with an input device.

In a further implementation of the first, second, and third aspects, the at least one patient parameter is indicative of a current medical state relative to a target medical outcome of the target patient, wherein the model is trained according to computed correlations with a current medical state relative to a target medical outcome associated with each of the at least one sample patients, and wherein the adaptation to the UI outputted by the model is computed for increasing likelihood of the current medical state reaching the target medical outcome.

In a further implementation of the first, second, and third aspects, the current medical state comprises a current value of the monitored patient parameter, and the target medical outcome comprises a target value or target range or target threshold.

In a further implementation of the first, second, and third aspects, the target medical outcome for the target patient is determined by correlating the respective at least one patient parameter and interaction journey to aggregated medical data collected from a plurality of subjects, and extracting the target medical outcome from the aggregated data.

In a further implementation of the first, second, and third aspects, the adaptation to the UI is selected from the group consisting of: zoom-in on a certain monitored patient parameter, marking a certain monitored patient parameter to attract attention of the healthcare provider, selection of a certain monitored patient parameter that is not presented in the UI for presentation in the UI, removal of a certain monitored patient parameter from the UI, relative positioning between two or more monitored patient parameters in the UI, presenting a message indicative of a recommendation for manual adaptation of the UI, presenting at least one aggregated medical data collected from a plurality of subjects, presenting suggested diagnoses in the UI, presenting a suggested treatment plan in the UI, and presenting one or more parameter of a customizable period of time in the UI, playing an audio message on speakers, presenting an augmented reality image on an augmented reality headset, and presenting a virtual reality image on virtual reality glasses.

In a further implementation of the first, second, and third aspects, further comprising: receiving an indication of an identity profile of the healthcare provider, feeding the identity profile of the healthcare provider into the model, wherein the model is trained on computed correlations according to identity profiles of the at least one sample healthcare providers.

In a further implementation of the first, second, and third aspects, the identity profile includes one or more members selected from the group consisting of: position, nurse, medical student, resident, staff physician, medical training rank, medical training, external and/or internal reviews, medical publication citations, medical specialty, demographic data, previous experience in performing medical procedures, medical skills, and success in treating other patients.

In a further implementation of the first, second, and third aspects, the at least one patient parameter is outputted by the at least one medical device.

In a further implementation of the first, second, and third aspects, the at least one patient parameter includes output of a plurality of physiological sensors that each measure a respective physiological parameter of the patient.

In a further implementation of the first, second, and third aspects, at least one patient parameter outputted by the plurality of physiological sensors are selected from the group consisting of: medical images, blood pressure measurement devices, arterial line, respiration devices, resuscitation devices, monitors, body temperature measurement devices, patient monitors, intracranial pressure sensors, Cerebral perfusion pressure sensors.

In a further implementation of the first, second, and third aspects, the at least one patient parameter are obtained from a plurality of non-physiological data sources that each store a respective non-physiological parameter of the patient.

In a further implementation of the first, second, and third aspects, the plurality of patient parameters obtained from the plurality of non-physiological data sources are selected from the group consisting of: patient demographics, identity profile of healthcare providing team members, history of the present illness, prior medical history, prior treatments, previously scheduled appointments, future scheduled appointments, treatment facilities where the target patient was treated.

In a further implementation of the first, second, and third aspects, the interaction journey and the at least one patient parameter are distributed to each of a plurality of processing nodes each hosting a respective model trained according to a unique computed correlations between interaction journeys of a plurality of unique healthcare providers with respective medical devices and the plurality of patient parameters of a plurality of unique sample patients, wherein outputs of the respective models are aggregated into an aggregated model and/or a single output of an adaptation to the UI.

In a further implementation of the first, second, and third aspects, further comprising feeding at least one aggregated medical data into the model, wherein the model is trained according to computed correlations between aggregated medical data and the interaction journeys of the at least one sample healthcare provider.

In a further implementation of the first, second, and third aspects, the aggregated medical data is selected from the group consisting of: medical research publication, and medical treatment guideline.

In a further implementation of the first, second, and third aspects, the at least one patient parameter is obtained from at least one care process data sources that each store a respective care process parameter of the patient.

In a further implementation of the first, second, and third aspects, the at least one care process data sources are selected from the group consisting of: which parameters each caregiver referred to, identity of treating caregiver, position of treating caregiver, expertise of treating caregiver, decisions of processes chosen by treating caregiver, actions of treating caregiver within the system, requests and referrals of treating caregiver, clicks, searches, time spent on each parameter, system overrides, corrections, re-checks, focuses of treating caregiver.

In a further implementation of the first, second, and third aspects, the adaptation to the UI comprises an alarm that is generated when at least one correlation is according to a requirement.

In a further implementation of the first, second, and third aspects, further comprising updating the model with at least one of a new correlation and/or a new adaptation to the UI provided by an external entity, and distributing the update to a plurality of instances of the model for execution using the updated model.

In a further implementation of the first, second, and third aspects, the at least one medical device comprises a plurality of medical devices, and the interaction journey of the healthcare provider is with the plurality of medical devices.

In a further implementation of the first, second, and third aspects, data sources of each at least one patient parameter are isolated and/or separated from other patient parameters by a specialized cybersecure network using security, cybersecurity, and/or network technology.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method for adapting a UI for presenting medical data for guiding treatment of a patient, in accordance with some embodiments of the present invention;

FIG. 2 is a block diagram of components of a system for adapting a UI, in accordance with some embodiments of the present invention;

FIG. 3 is a block diagram of another exemplary system for adapting a UI for presenting medical data for guiding treatment of a patient, in accordance with some embodiments of the present invention;

FIG. 4 is a block diagram of yet another exemplary system for adapting a UI for presenting medical data for guiding treatment of a patient, in accordance with some embodiments of the present invention;

FIG. 5 is a dataflow diagram of an exemplary dataflow for correlating patient medical data and other data, in accordance with some embodiments of the present invention;

FIG. 6 is another dataflow diagram of an exemplary dataflow for correlating patient medical data and other data, in accordance with some embodiments of the present invention;

FIG. 7 is a schematic of a UI presenting patient parameters for a monitored patient, in accordance with some embodiments of the present invention;

FIG. 8 is a schematic depicting an example of a UI implemented as a display presenting multiple patient parameters, in accordance with some embodiments of the present invention;

FIG. 9 is a schematic depicting exemplary data sources that stores and/or output data for computation of the patient parameters (labeled as micro-information), and exemplary aggregated data source (labeled as macro-information), which are fed into the model with the interaction journey for outputting the adaptation of the UI, in accordance with some embodiments of the present invention;

FIG. 10 is a high level data flow depicting the process of analyzing output of the model fed the interaction journey and patient parameters, in accordance with some embodiments of the present invention;

FIG. 11 is a schematic depicting an application system and/or application programming interface (API) for interacting with external entities, in accordance with some embodiments of the present invention;

FIGS. 12A-E are exemplary schematics of the UI adapted based on output of the model fed the interaction journey and patient parameter(s), in accordance with some embodiments of the present invention;

FIG. 13 is a schematic of an exemplary workflow alert and/or exemplary alarm outputted by the model, in accordance with some embodiments of the present invention;

FIG. 14 is a schematic of an automated correlation between a marking on a 2D image and a corresponding computed marked location on and positioning of a 3D image, in accordance with some embodiments of the present invention; and

FIG. 15 is a schematic of a UI presented on a screen that is split into a data window and an image window, where the image is marked with a marking by a user, in accordance with some embodiments of the present invention.

### DETAILED DESCRIPTION

The present invention, in some embodiments thereof, relates to the technical field of analysis of medical data of a patient and, more specifically, but not exclusively, to adaptation of a UI based on an analysis of medical data of a patient.

As used herein, the term healthcare provider may be exchanged with the term user. The healthcare provider is the user of the system, for example, the healthcare provider may use the system for planning treatment of a patient.

An aspect of some embodiments of the present invention relates to systems, methods, apparatus, and/or code instructions for adapting a user interface (UI) for presenting medical data of a target patient, for example, for assisting the healthcare provider using the UI in treating the patient, planning treatment of the patient, and/or monitoring the health status of the patient. An interaction journey of the healthcare provider with one or more medical devices is monitored. The medical devices store data of the target patient and/or monitor the target patient and/or are used for treatment of the patient, for example, blood pressure sensor, computing device presenting an electronic medical record, viewer application installed on a computer device accessing medical images stored on an imaging server and/or software system, patient room monitor, operating room monitor, and ventilation machine. The interaction journey denotes, for example, what buttons the healthcare provider pressed on the medical device, the sequence of actions the healthcare provider performed on the medical device, what data the healthcare provider selected for viewing on the medical device, and what data the healthcare provider entered via the medical device. One or more patient parameters of the target patient are monitored. Patient parameters may be, for example, physiological measurements obtained by sensors (e.g., blood pressure, temperature, intracranial pressure), and/or other stored data (e.g., patient identity, profile of the healthcare provider, medical images, medical diagnosis stored in the EMR). The interaction journey and one or more patient parameters are fed into a model (e.g., machine learning model, machine learning classifier, statistical model, etc.) that is trained according to computed correlations, computed interactions, computed differences, etc., between interaction journeys of multiple other sample healthcare providers (and optionally the current healthcare provider) with respective medical devices and one or more patient parameters of one or more other patients (and optionally the current patient). The model outputs an adaptation to the UI, for example, presenting data of the patient determined to be relevant not currently presented on the UI, an alert, and/or a recommendation for treatment.

At least some implementations of the systems, methods, apparatus, and/or code instructions described herein relate to the technical problem of analyzing a large amount of collected medical data, including aggregated data from other patients, physiological data of the patient collected by sensors, and non-physiological data of the patient. Analyzing the large amount of material is challenging to users, for example, due to the limited amount of time they have and the difficulty in identifying which values of which data is significant. At least some implementations of the systems, methods, apparatus, and/or code instructions described herein provide a UI that is fed an interaction journey of a user with medical device(s) and is fed one or more patient parameters. The model outputs instructions for adapting the UI, based on learned interactions by other users with different medical devices for other patients with correlated patient parameters. The adapted UI aids the user in analyzing the large amount of data, for example, by presenting the data determined to be the most relevant data, and/or presenting suggestions and/or alerts.

At least some implementations of the systems, methods, apparatus, and/or code instructions described herein relate to the medical problem of treating a patient based on a large amount of collected medical data, including aggregated data from other patients, physiological data of the patient collected by sensors, and non-physiological data of the patient. Analyzing the large amount of material in order to generate a treatment plan is challenging to physicians and/or other healthcare workers, due to the limited amount of time they have and the difficulty in identifying which values of which data is significant. At least some implementations of the systems, methods, apparatus, and/or code instructions described herein provide a UI that is fed an interaction journey of a user (e.g., physician and/or nurse planning medical treatment) with medical device(s) and is fed one or more patient parameters. The model outputs instructions for adapting the UI, based on learned interactions by other users with different medical devices for other patients with correlated patient parameters. The adapted UI helps the physician to better plan the treatment, for example, by presenting the data determined to be most relevant data, and/or presenting suggestions for treatment.

At least some implementations of the systems, methods, apparatus, and/or code instructions described herein improve the technology of a UI, by improving the ability of the UI to present relevant medical data. The space available on the display, presented by the UI, is limited, while the amount of medical data available for the patient is very large. All the data cannot be presented within the UI at the same time. A decision needs to be made when to present which data, in order to help monitor and/or treat the patient. Traditionally, physicians made such decisions manually, based on their prior experience and/or medical training. At least some implementations of the systems, methods, apparatus, and/or code instructions described herein automatically and dynamically adjust the UI, for example, to present the most relevant data for the current patient based on monitored patient parameter(s), and/or based on learned interactions of other physicians with other medical devices associated with other patients. Such adjustments improve the utilization of the limited available space of the UI by at least automatically presenting the medical information that is most relevant to planning treatment of the target patient. Such adjustments improve the ability of the physician to plan the treatment.

Furthermore, much can be learned about the treatment process by observing and recording the treatment process electronically, which is enabled by the present invention.

At least some implementations of the systems, methods, apparatus, and/or code instructions described herein assist the healthcare provider in monitoring the patient and/or treating the patient, based on learned interactions correlated with patient parameters acted upon by the same and/or other healthcare providers in similar situations (i.e., as outputted by the model). Some examples are now provided:

For a patient with a certain medical diagnosis, the majority of physicians check the latest blood test results, check recent x-rays, and adjust medication accordingly. For a target patient diagnosed with the certain medical condition, and when the current healthcare provider has started the interaction journey with checking the latest blood test results, the model outputs instructions for presenting on the UI a message indicating no recent x-rays, and presenting a window for adjusting patient medications. The adjustments to the UI based on the output of the model save time for the physicians (i.e., instead of manually logging to check the PACS and logging into the medication system) and/or help ensure the physician does not forget certain parts of the treatment flow (e.g., forget to check x-rays and/or forget to adjust medications) and/or helps direct medical students as to what to do next by helping walk the medical student through interaction journeys that are common medical practice of other physicians in similar clinical situations.

For a patient for which blood tests are collected every day, and for which the interaction journey is the checking of the blood test results, the model may output an alert for presentation on the UI recommending a change in medication when the blood test values have changed but are still within normal limits (or when blood tests results are consistently abnormal but now require attention), based on computed correlation between interaction journeys of hematology specialists that changed the medication and other similar changes in blood test results for similar patients. The model enables, for example, non specialist physicians to be made aware of clinical practice of specialist(s), to improve treatment of the patient without requiring constant involvement of the specialist. Since the change in blood test results may be still within normal (or abnormal values), a non-specialist that is not specifically looking for the change in value may not be aware of the significance of the change using standard systems, for example, that only alert when values are outside of normal range. It is noted that the model may be fed the identity profile of the treating healthcare provider, indicating whether the respective healthcare provider is a specialist or not, the respective healthcare provider's level of experience, the respective healthcare provider's success in treating similar cases, etc. The model may output the alert to non-specialists and not output the alert for specialists that may have a history of such changes in medication for similar changes in blood tests results.

In another example, a model learns the correlation between an interaction journey of making certain changes to a ventilator and a note in the EMR of the patient by a specialist that the patient should be prepared for removal of the endotracheal tube. When a user makes the same changes and no such note is in the EMR, the model outputs instructions to present a window to call for the specialist to evaluate the patient for removal of the tube.

Additional examples are described below.

At least some implementations of the systems, methods, apparatus, and/or code instructions described herein relate to the technical problem of processing a large amount of medical information for selecting relevant data for treatment of a patient. The digitization of medical equipment and data has been a growing area of interest in the last several decades. Attempts were made to collect data from medical monitors, respirators, and other vital equipment that is used in hospitals and physicians' offices and clinics, etc. Such equipment is, for example, but is not limited to, blood pressure measurement devices, respiration devices, resuscitation devices, monitors, body temperature measurement devices, and other similar and different devices used for the purpose of the provision of medical care, scheduling of appointments, choosing the appropriate and correct providers and facilities, and other purposes in support of improving and making more efficient a medical practice, hospital, medical clinic, ambulance, medical transport, and any other medical applications. Recently, a variety of computerized mathematical and digital processes have been developed to try and analyze the copious amounts of data that is being collected digitally in a wide variety of systems from various applications and industries. However, existing systems and/or methods are focused on a single data source for a specific patient, and/or a single desired output, and cannot cope with large amounts of different data for helping improve the overall planning of treatment of a patient. Existing systems and methods of computerized mathematical and digital processes look only at a limited set of data. For example, a system that collects medical images (e.g., X-rays, CT scans, MRI images) is designed for the purpose of identifying single data items such as a fracture, a cancerous growth, or general features such as any anomalies that are discovered in the image, using machine learning or other strategies. Such imaging systems only provide images for specific patients and may only provide an analysis of findings in those images for specific patients. In another example, an app that collects data about a patient's symptoms and identifies appropriate treatments or remedies using computerized mathematical and digital processes or other strategies is limited to the patient's ability to describe and enter their own symptoms, while other data cannot be provided and considered. Using multiple different systems, providing different data about the patient in order to obtain an accurate and complete picture of the state of the patient and plan the treatment is difficult and time consuming for healthcare providers, even and especially using existing systems. Furthermore, in the existing systems, it is not possible for physicians to follow the care process and decisions of others. Huge amounts of interesting data is lost due to this lack of a lens into the care process. In contrast, at least some implementations of the systems, methods, apparatus, and/or code instructions described herein consider, in addition to standard medical data of the patient, other rich datasets, such as the interaction journey with one or more medical devices of physicians, nurses, and caregivers, which may be indicative of the work process and/or treatment planning process, and optionally other information, as described herein. At least some implementations of the systems, methods, apparatus, and/or code instructions described herein improve the technology of medical information management, by performing one or more of: collecting patient parameters from various sources, monitoring an interaction journey of the health care provider(s) with one or more medical devices, analyzing the data in real time by the model that receives the interaction journey and patient parameters, and outputting by the model instructions for adapting a UI for visualizing data in a clear and readable way in front of the medical team (e.g., nurses, doctors, clinician doctors, surgeons, pharmacists, etc.) so the medical personnel is able to provide the best possible treatment to the patient on hand, and develop a real time battle plan for immediate medical needs as well as for long term needs.

At least some implementations of the systems, methods, apparatus, and/or code instructions described herein collect and analyze data which is not traditionally considered, including at least the interaction journey of the user with one or more medical devices associated with the patient (e.g., monitor the patient, storing data of the patient), in contrast to traditional systems and methods that consider data for a single patient, and/or of a single type (e.g., imaging) and/or of a single or specific outcome (e.g., detect cancer in images). Such systems do not consider the interaction journey of the user with medical devices and/or other information systems and/or the systems themselves.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive (SSD), a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language, Python programming language, or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

At least some implementations of the systems, methods, apparatus, and/or code instructions described herein are related to the medical field and analysis, collection and aggregation of disparate data in novel and any other creative ways, and the use of novel computerized mathematical and digital processes and algorithms on medical data for the benefit of practitioners, physicians, surgeons, clinicians, nurses and other caregivers and to improve care for patients.

At least some implementations of the systems, methods, apparatus, and/or code instructions described herein provide a platform that federates and tracks medical data, information, and details about the full scope of the treatment process. The model described herein analyzes patient parameter(s) and an interaction journey of a user with medical devices storing data of the patient and/or monitoring the patient, optionally with other contextual data, as defined herein, and may analyze them simultaneously.

At least some implementations of the systems, methods, apparatus, and/or code instructions described herein is related to the medical field and/or provides analysis and/or the use of computerized mathematical and/or digital processes on various medical data. The medical data is collected by processes that may enable a real time analysis and/or display of results, for example, on a special, inseparable, and/or unified input and output system that is comprised of, for example, touch screens, screens, monitors, tablets, smart phones, keyboards, mice, dials, wheels, digital pen tablets, digital and analog pens, speakers, cameras, headphones, VR systems, VR glasses, cables, etc. The system may collect, input, output, visualize, and/or interpret the data and/or provide a holistic overview of a medical situation in real time for the benefit of medical personnel and patients alike. The system may collect richer data beyond the patient medical data that is collected by existing systems.

As used herein, the term point data may sometimes refer to patient medical data, in particular, physiological measurement of the certain target patient for example, medical images, patient blood tests, and patient blood pressure.

As used herein, the term contextual data may sometimes refer to non-physiological measured data of the target patient and/or other data that may be indirectly related to treatment of the patient, in particular, at least the interaction journey of the healthcare provider with one or more medical devices storing data of the target patient and/or monitoring the target patient, and/or other contextual data, for example, clinical information, electronic medical record data, medical images, pharmacy record, a profile of the user (e.g. treating healthcare worker), environmental conditions, medical guidelines, nursing guidelines, other guidelines, medical publications, nursing publications, other publications, genetic findings, results, and research, and/or research in medicine, chemistry, biology, psychology, other scientific or non-scientific fields that may be relevant to the treatment of the present patient.

At least some implementations of the systems, methods, apparatus, and/or code instructions described herein correlate and/or combine “Point data” and “contextual data” using computerized mathematical and/or digital processes and/or other machine learning processes such as but not necessarily limited to statistics, mathematics, artificial intelligence, machine learning and/or computer science based processes to learn, analyze, and/or understand the condition of the patient on hand, and/or how and/or where to treat the patient in a given situation.

At least some implementations of the systems, methods, apparatus, and/or code instructions described herein provide a way to gain an insight, overall scope, from clinical and/or biomedical data and/or information collected for a specific patient. At least some implementations of the systems, methods, apparatus, and/or code instructions described herein provide personalized medicine and/or healthcare specified to the benefit of each patient. At least some implementations of the systems, methods, apparatus, and/or code instructions described herein provide an engineering system constituted from individual or general input devices which are decided according to the needs and desires of the facility it is used in and/or previous experience in similar facilities, and/or algorithms which aid in the decision of such inputs and outputs.

At least some implementations of the systems, methods, apparatus, and/or code instructions described herein are unlike standard processes, by considering multiple data from various disparate systems. This includes the results of existing machine learning systems as well as measurement devices, as described herein. In contrast to traditional processes that are focused on discovering a particular result of a particular question regarding a patient, a particular business insight, or discovering unanticipated conditions about a patient, at least some implementations of the systems, methods, apparatus, and/or code instructions described herein provide a platform to analyze and/or federate multiple data and/or results combined to give a holistic overview insight of a given treatment that stems from previous experiences and/or a holistic view of such previous experiences. In addition, non-traditional “contextual data” regarding which caregiver is performing the treatment, their skills, experiences, and success at treating previous patients, may be considered by being fed into the model. In addition, a specific subset of “contextual data”, the interaction journey, may be considered by being fed into the model. Outcomes of treatments and/or the caregivers, the hospitals, and similar facilities may be considered by being fed into the model. Other sources of appropriate information may be considered by being fed into the model.

Optionally, “point data” and “contextual data” are correlated and/or combined using computerized mathematical and digital processes (e.g., the model) and/or other processes to learn, analyze, and understand, etc., the treatment plan for the patient and how to improve the plan.

Optionally, “point data” and “contextual data” are correlated and/or combined using computerized mathematical and digital processes (e.g., the model) and/or other processes to learn, analyze, and understand, etc., the performance of nurses, physicians, and other caregivers for the patient, and to suggest the most appropriate physicians, nurses, and other caregivers.

Optionally, “point data” and “contextual data” are correlated and/or combined using computerized mathematical and digital processes (e.g., the model) and/or other processes to learn, analyze, and understand, etc., the performance of hospitals, clinics, facilities, and other medical organizations, and to suggest the most appropriate hospitals, clinics, facilities, physicians' offices, and medical organizations for the given scenario.

Optionally, “point data” and “contextual data” are correlated and/or combined using computerized mathematical and digital processes (e.g., the model) and/or other processes to learn, analyze, and understand, etc., business insights regarding the performance, operations, quality, etc. of the hospital, clinic, or medical organization. Optionally, “point data” and “contextual data” are correlated and/or combined using computerized mathematical and digital processes (e.g., the model) and/or other processes to learn, analyze, and understand, etc., the provision of similar treatments to previous situations, and correlate activities, diseases, disorders, treatment strategies, etc. in a way not currently performed. By looking at large amounts of past data, research data, etc., better and more appropriate treatments for patients may be found.

At least some implementations of the systems, methods, apparatus, and/or code instructions described herein correlate data from multiple instances (e.g., of the computing device), including when implemented in disparate locations. This may be done securely and in a way that maximally maintains privacy, and only with the permission of the relevant institutions and individuals.

At least some implementations of the systems, methods, apparatus, and/or code instructions described herein uses computational and/or computerized techniques and/or computerized mathematical and/or digital processes as described herein to get a full scope of the patient's condition based on many physiological conditions with high frequency collection, and in real time display a treatment plan for the physicians, clinicians, surgeons, nurses, and other caregivers.

At least some implementations of the systems, methods, apparatus, and/or code instructions described herein uses a wide scope, and looks at many aspects of the treatment, for example but not limited to medications that were prescribed to the patient, data that was collected from various equipment, the personnel that examine this particular patient, their experiences, skills, etc. the physicians' train of thoughts as reflected on tests, medications, assumptions, way of examination, etc., and calculate the best treatment plan as they reflect on those data particularly combined with the extensive previous data that was analyzed by the system.

At least some implementations of the systems, methods, apparatus, and/or code instructions described herein are built from the components described herein, analyzes and/or provides an overview and insight on the entire medical treatment process that is derived from the basic data (e.g., patient parameters) and contextual data (e.g., at least the interaction journey), and optionally historical medical treatments that are analyzed from the contextual data, optionally for the same given medical condition.

At least some implementations of the systems, methods, apparatus, and/or code instructions described herein provide a general overview and/or insight of the treatment process. For example, but not necessarily limited to, zoom in on previous treatment processes and/or check which measures were used and by whom: a physician, clinician, nurse, etc., and this person's experience, to analyze the specific medical condition of the patient. Based on this specific condition, the model may learn which are the necessary steps needed to be performed for such condition and present the results (by adapting the UI) to the current caregivers. In turn, the caregivers on hand may check themselves, visualize by looking at the screen's presentation of the UI which steps are taken for this condition in previous cases, for example, but not necessarily limited to what medications are prescribed, where are the checkpoints, etc. and thus provide more efficient treatment to the patients on hand. This data, that consists of point data (i.e., patient parameters) and/or contextual data (e.g., at least the interaction journey) analysis, provides a novel insight and complete battle plan for treating the patient.

At least some implementations of the systems, methods, apparatus, and/or code instructions described herein provide treatment analysis based on contextual data (e.g., at least the interaction journey) and/or the point data (i.e., patient parameters) that are combined. At least some implementations of the systems, methods, apparatus, and/or code instructions described herein unify the relevant data and/or information that is connected to a certain condition and enables the caregiver on hand to get in real time the detailed overview, for example, by displaying on a touch screen and/or other screens. This includes but is not limited to the caregiver's zoom in preferences, checkup, and medication preferences, tests and images, etc. The model learns from the past from data available and/or shows, for example but not necessarily limited to treatments, medical thoughts and/or notes, medication to the on hand caregiver for the benefit of the on hand patient.

At least some implementations of the systems, methods, apparatus, and/or code instructions described herein tracking the interaction and/or the contextual data and/or in the basic data and/or tracing and/or tracking the caregiver activities, order of operation, and/or their train of thoughts. For example, but not necessarily limited to, where and/or on what images the caregiver clicked, which measurement were essential to a treatment decision, which lab test were ordered in cases similar to the given case, identity and/or experience of the caregiver, demographic note of the patients, etc. (“the interaction journey”).

At least some implementations of the systems, methods, apparatus, and/or code instructions described herein contain a specialized cybersecure network that isolates and/or separates the different sources of point and/or contextual data, using network technology including for example virtual local area networks (VLAN's), virtual extensible local area networks (VXLAN's), encapsulation, packet encapsulation, packet inspection, deep packet inspections, firewalls, virtual networks, software defined networks (SDN's) including SDN-enabled networking software and hardware hubs, switches, routers, and other networking components, overlays, underlays, terminal access points (TAP's), tunnels (TUN's), wired networks, ethernet networks, wireless networks, and other appropriate networking technologies. The specialized cybersecurity network may improve security of the data sources, for example, maintaining privacy of the data sources and/or reducing risk of malicious interception and/or use of the data sources.

Optionally, the outputted information and/or analysis are visualized and/or displayed on the invention's output media that are for example but not necessarily limited to the invention's touch screen, pen, dial, wheel, screen, display, VR system, VR glasses, speakers, headphones, etc.

Optionally, after collecting and/or storing the data from several medical devices (i.e., the interaction journey and/or patient parameters) that fits an individual target patient, and that is varied as needed from patient to patient as explained herein, the data may be analyzed by applying the model (e.g., a state of the art analysis and/or insights process) to give a real time overview of the individualized patients conditions and/or needs. By connecting to all relevant data from medical equipment that referred to the individual patients, the overall state of this individual patient is tracked and thus general overall insight on the patient condition for a certain time interval may be provided. Each time interval may be registered, saved and/or stored and/or the medical providers may go back and forth to see the relevant data in each given moment or other time frame.

At least some implementations of the systems, methods, apparatus, and/or code instructions described herein combine data collection, data consumption/visualization and/or data analysis and/or processes that together provide insight to practitioners, doctors, surgeons, clinicians, nurses and/or other caregivers, for example, to create a novel “battle plan” and/or treatment plan, treatment schedule, new medication and/or other such treatment possibilities for the benefit of the patient, and over a user-customizable scale of time from milliseconds to years.

At least some implementations of the systems, methods, apparatus, and/or code instructions described herein personalize and/or accumulate and/or store the collected data in real time for each patient separately. This data may include all data from, for example but not necessarily limited to, monitors and/or medical equipment that is connected to that specific patient. Charts, maps, diagrams may be customized to the specific use. The data may be stored and/or saved, and/or it is available to use and/or reused at a click of a button.

At least some implementations of the systems, methods, apparatus, and/or code instructions described herein provide a clear, consistent, and/or customizable user interface (that is optionally adapted by the output of the model described herein) that fits well into the existing workflow at healthcare facilities such as but not necessarily limited to, intensive care units, such as but not necessarily limited to inpatients units/departments/offices or other provider's institutions that adapt to individual users. At least some implementations of the systems, methods, apparatus, and/or code instructions described may be customized to each such user and/or patient.

At least some implementations of the systems, methods, apparatus, and/or code instructions described herein utilize the interaction journey and/or patient parameters and/or other data, which are fed into the model, to alert or alarm to the user exceptional conditions regarding the patient, or activities that need to be performed to better care for the patient, for example, but not limited to, for the reduction of infection or for the reduction of pressure ulcers.

At least some implementations of the systems, methods, apparatus, and/or code instructions described herein utilize the interaction journey and/or patient parameters and/or other data, which are fed into the model, optionally with other optimization techniques to provide clean data and/or predict and/or show the condition of interest without necessarily causing alarm fatigue.

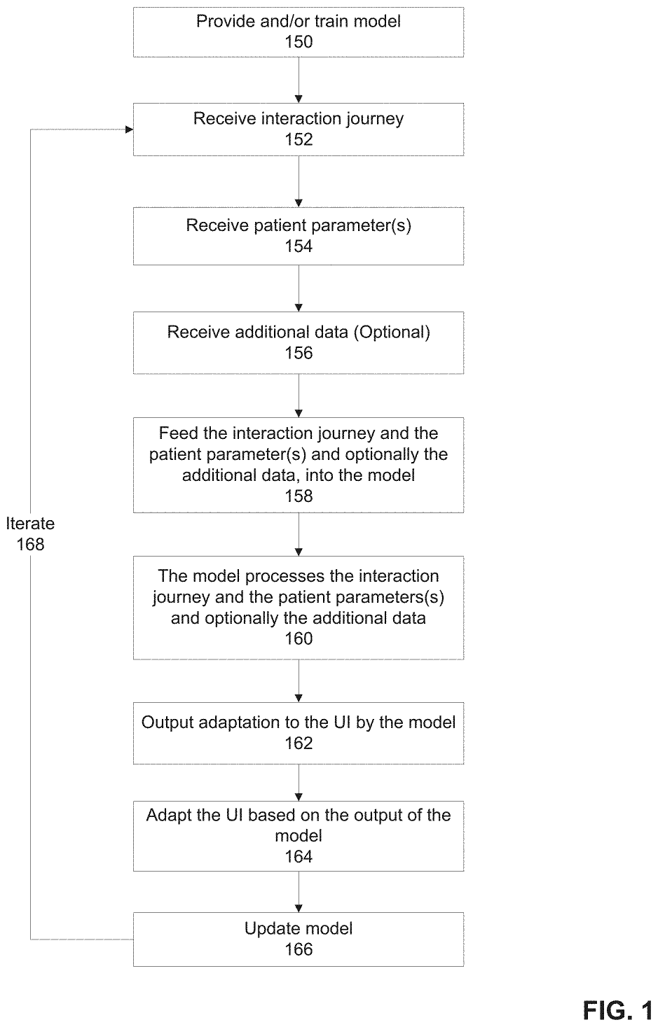

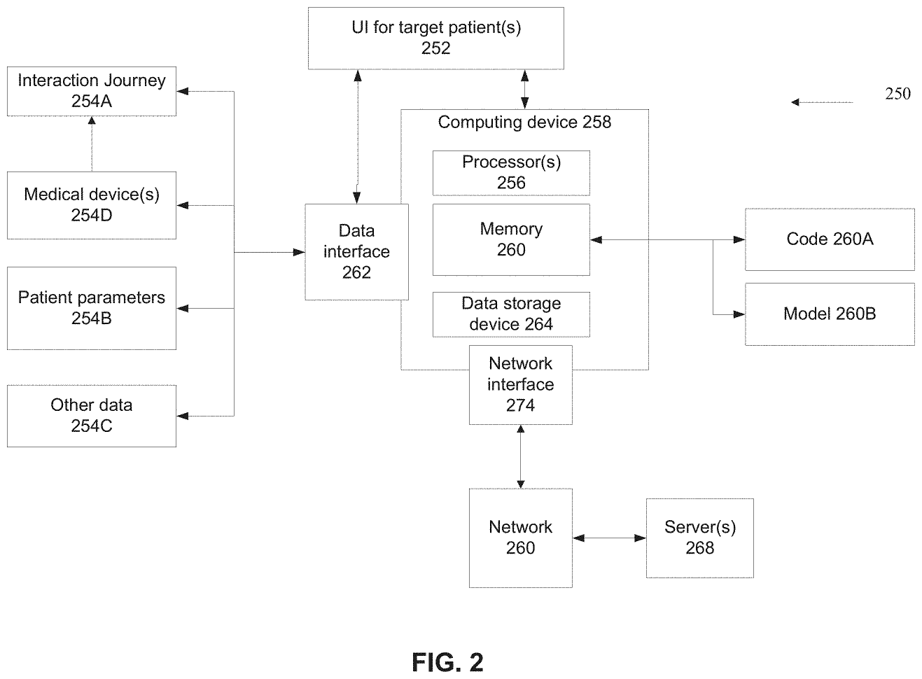

Reference is now made to FIG. 1, which is a flowchart of a method for adapting a UI for presenting medical data for guiding treatment of a patient, in accordance with some embodiments of the present invention. Reference is also made to FIG. 2, which is a block diagram of components of a system 250 for adapting a UI 252, in accordance with some embodiments of the present invention. UI 252 may present medical data for guiding treatment of a patient. One or more acts of the method described with reference to FIG. 1 may be implemented by components of system 250, as described herein, for example, by a hardware processor(s) 256 of a computing device 258 executing code instructions 260A stored in a memory (also referred to as a program store) 260.

Code 260A and/or model 260B may implement one or more of the following exemplary technologies (not necessarily limited to): computer code, such as a web interface, Javascript, Java, Objective C, Swift, HTML, CSS, C, C++, Python and/or other software programming languages and/or tools may be used to implement machine learning strategies and algorithms in the system.

Computing device 258 may receive data via one or more data interfaces 262. Data may include one or more of:

An interaction journey 252A denoting an interaction of a user with one or more medical device(s) 254D. The interaction journey 252A may be computed, for example, by hardware on the respective medical device 254D, by computing device 258, and/or by another device. Exemplary medical devices 254D are described herein.

Patient parameter 254B of the patient. Exemplary patient parameters are described herein, and include, for example: Physiological data (e.g., measurements) outputted by physiological sensor(s) sensing target patients. Physiological data is collected per target patient. Exemplary physiological sensors include: patient monitors, patient respiration devices, blood pressure measurement device, resuscitation device, body temperature device, intracranial pressure sensor, and cerebral perfusion pressure sensor. Patient parameters may include non-physiological data stored by non-physiological data sources. Non-physiological data is stored and/or provided per target patient. Exemplary non-physiological data include: identity of the patient, demographic data of the patient.

Other data, as described herein. Exemplary other data includes: Aggregated data obtained from a medical data source storing data aggregated from multiple patients, for example, published medical studies, clinical guidelines, and/or hospital guidelines. The aggregated data may be stored, for example, on a local storage device, a web server, a computing cloud, and/or received over a network (e.g., as an email, and/or other subscription service, and/or connected from a different medical system and/or device). Other data may include a profile of the healthcare provider, for example, medical specialty and/or year of service and/or rank.

Data sources 254A-D may be dynamically updated and provided to computing device 258, for example, continuously (e.g., output of a pulse oximeter) and/or per event (e.g., as newly published studies are available).

Computing device 258 analyzes the received data and generates instructions for adapting UI 252, as described herein.

UI 252 may be implemented as, for example, as large high resolution screen, optionally touch screen, a virtual reality glasses, an augmented reality head gear, speakers playing audio, headphones, a dedicated screen of a medical device, a screen of a smartphone, and a screen of a computer. UI 252 may be implemented as a GUI. Device 252 may be designed to enable a user to interact with the displayed UI, for example, a pen designed to provide input to a computer, a dial, a wheel, a keyboard, a mouse, augmented reality glasses, voice activated software using speakers, a headset, and/or microphone, and a camera capturing images of the user interaction. Alternatively, display 252 is designed only for data presentation without interaction ability, for example, to present monitored patient data, and/or alerts. User interface 252 may include mechanism(s) for providing output, for example, a display, a system of displays, glasses virtual reality or augmented reality glasses, and/or speakers. Computing device 258 may be in communication with user interface 252 and/or another user interface.

UI 252 may be designed for displaying image(s) in a 2D and/or 3D and/or 4D form, and/or rotate the image, for example, 360 degrees. This capability may enable the doctor, physician, clinician, surgeon, nurse, pharmacist, and/or other caregiver personnel the possibility to check, for example but not necessarily limited to, imaging and charts simultaneously in a real time and customized format, adjust or replace battle plan for the individual patient.

User interface 252 may include one or more input devices and/or input components. User interface 252 (e.g., input component thereof) may be designed for users (e.g., physicians, nurses, other caregivers, as well as patients, visitors, and other individuals) interacting and/or inputting information. For example, but not necessarily limited to: a large touch screen display, a pen that is capable of writing on the touch screen, a dial that may be placed on the screen and provides interactivity, a keyboard that allows users to type words, a camera, a microphone, glasses with virtual reality (VR) or augmented reality (AR) capability, and/or ports that allow the input of data from other systems, such as USB ports, a wireless networking card, and/or an Ethernet port. Data as described herein may be entered automatically and/or manually. Such information may be confirmed by caregivers of the patient. In addition, software such as a web interface, Javascript, Java, Objective C, Swift, HTML, CSS, C, C++, Python, NodeJS and/or other software programming languages and/or tools may be used to input and/or confirm data and information in the system.

User interface 252 may include one or more output devices and/or output components. The user interface 252 (e.g., output component thereof) may be designed for users (e.g., physicians, nurses, other caregivers, as well as patients, visitors, and other individuals) interacting and/or retrieving information. For example, but not necessarily limited to: a large touch screen display, a pen that is capable of writing on the touch screen, a dial that may be placed on the screen and provides interactivity, a keyboard that allows users to type words, a camera, a microphone, glasses with virtual reality (VR) or augmented reality (AR) capability, a large screen and port that allow the output of data to other systems, such as USB ports, a wireless networking card, and/or an Ethernet port. On the output components, data as described herein may be extracted. The information may be presented in a way that is familiar to caregivers and matches the current flow of their work. In addition, software such as a web interface, Javascript, Java, Objective C, Swift, HTML, CSS, C, C++, Python, NodeJS and/or other software programming languages and/or tools are used to output data and information in the system.

Data interface(s) 262 may be implemented, for example one or more of: a network interface, a port, a direct link, a wire connection, a wireless connection, a local bus, other physical interface implementations, and/or virtual interfaces (e.g., software interface, application programming interface (API), software development kit (SDK)).

Computing device 258 may be implemented as, for example, a standalone unit, a dedicated device, a smart television integrated with display 252, a client terminal, a server, a computing cloud, a mobile device, a desktop computer, a thin client, a Smartphone, a Tablet computer, a laptop computer, a wearable computer, glasses computer, a watch computer, a virtual machine, a virtual server, Arduino device (or similar or different), a mini-computer, a sensor network, a “mote”, and a backpack-computer.

Different architectures of system 250 may be implemented. For example:

Computing device 258 is provided per target patient. For example, each target patient is provided with a bedside display 252. Computing device 258 is connected to data sources 252A-D, and generates instructions for adaptation of UI on display 252. Computing device 258 may be integrated with display 252, for example, as a smart television, or in communication with display 252, for example, as a computing device connected to display 252.

Computing device 258 may be implemented as a server providing services to multiple displays 252. Each patient is provided with a dedicated display 252. Data sources 252A-D are collected for multiple target patients are fed to computing device 258, which outputs instructions per display 252.

A single display 252 is provided for multiple target patients. A dedicated computing device 258 is implemented for each display 252, or a server computing device 258 provides services to multiple displays 252 each used for multiple target patients. For example, display 252 is implemented as a portable station and/or cart, which is moved from room to room as physicians perform rounds. The UI for each target patient is loaded when the physicians are visiting that patient. Or, in another example, the display 252 is mounted in a centralized location in the hospital and displays the information of multiple patients simultaneously.

Hardware processor(s) 256 may be implemented, for example, as a central processing unit(s) (CPU), a graphics processing unit(s) (GPU), field programmable gate array(s) (FPGA), digital signal processor(s) (DSP), and/or application specific integrated circuit(s) (ASIC). Processor(s) 252 may include one or more processors (homogenous or heterogeneous), which may be arranged for parallel processing, as clusters and/or as one or more multi core processing units.

Memory (also known herein as a data storage device) 260 stores code instructions executable by processor(s) 256, for example, a random access memory (RAM), read-only memory (ROM), and/or a storage device, for example, non-volatile memory, magnetic media, semiconductor memory devices, hard drive, solid state drive, removable storage, and optical media (e.g., DVD, CD-ROM). Memory 260 stores code instruction 260A that implement one or more acts of the method described with reference to FIG. 1. Memory 260 stores a model 260B that outputs the instructions for adapting the UI, as described herein. Alternatively, or additionally, one or more acts of the method described with reference to FIG. 1 are implemented in hardware.

Computing device 258 may include a data storage device 264 for storing data, for example, the received data. Data storage device 264 may be implemented as, for example, a memory, a local hard-drive, a local solid state drive, a removable storage unit, an optical disk, a storage device, and/or as a remote server and/or computing cloud (e.g., accessed via a network connection).

Computing device 254 may include a network interface 274 for connecting to network 260, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 254 may access one or more remote servers 268 using network 260, for example, to provide and/or obtain data from other remotely located computing devices, for example, from another model located at another medical facility that is trained based on data collected from the other medical facility, as described herein, to download updated code 260B, and/or model code 260B.

It is noted that data interface 262 and network interface 274 may be implemented as a single interface (e.g., network interface, single software interface), and/or as two independent interfaces such as software interfaces (e.g., as APIs, network ports, virtual network ports) and/or hardware interfaces (e.g., two network interfaces), and/or combination (e.g., single network interface, and two software interfaces, two virtual interfaces on a common physical interface, virtual networks on a common network port). The term/component data interface 262 may sometimes be interchanged with the term network interface 274.

Data interface(s) 262 and/or network interface 274 may be implemented as a communications component, for communicating with other nodes and/or instances of the system and/or external nodes. For example, but not necessarily limited to: USB ports, wireless networking card, an Ethernet port, and/or instructions for a human user to transfer information between different nodes of the system, and/or other similar or different tools to achieve this purpose. In addition, software such as a web interface, Javascript, Java, Objective C, Swift, HTML, CSS, C, C++, Python, NodeJS, and/or other software programming languages and/or tools may be used to implement the communications in the system.

Referring now back to FIG. 1, at 150, a model is trained and/or provided. The model is trained according to computed correlations between interaction journeys of one or more subject healthcare providers who may be employed by the same or different, separate, and non-cooperating institutions with respective medical devices and one or more patient parameter of one or more subject patients. The model may be trained based on the interaction journey of the current healthcare provider and patient parameter(s) of the current patient.

Alternatively or additionally, the model is trained according to computed correlations between the interaction journeys of the current healthcare provider and one or more patient parameters of the current target patient. In this manner, the model may be customized per patient or by the patient, by learning the interactions for the user treating the specific patient.

The model may be trained, for example, centrally for use by multiple healthcare providers for treatment of respective multiple patients, personalized per healthcare provider, personalized per patient, and/or combinations thereof.

The correlations may be a multi-dimensional space, where the interaction journey is one or more dimensions, and each patient parameter denotes one or more respective dimensions.

The output of the model, in the form of instructions for adaptation of a UI, may be based on achieving a target. The target may be automatically computed by code, for example, when a certain interaction journey is correlated with a certain patient parameter, the next action taken by the healthcare provider(s) (i.e., the next step in the interaction journey) may be automatically designated as the target output of the model. In such implementation, the model outputs the next action previously taken by the healthcare provider(s) as the adaptation to the UI when fed the certain interaction journey and the certain patient parameter. The target may be manually defined by a user, for example, a healthcare provider may perform a certain interaction journey which is correlated with a certain patient parameter. The healthcare provider may manually set the target, for example, define a certain value of the patient parameter as a goal. In such a case, when the model is fed the certain interaction journey and the certain patient parameter an alert may be generated stating that the certain value is the goal.

Optionally, the model is trained according to computed correlations with a current medical state relative to a target medical outcome associated with the sample patients. For example, a target value of a target patient parameter (e.g., blood pressure below a certain value, HbA1c target range), and/or a target medical outcome such as: elimination of a certain medication, removal of a certain diagnosis, discharge from hospital, and avoidance of a surgery. The target medical outcome may be automatically detected and/or manually defined by a user. The model may be trained to generate output which a relatively higher likelihood of reaching the target medical outcome. For example, when fed a certain interaction journey and certain patient parameter(s), the output (e.g., presentation of a certain patient parameter, suggestion to provide a certain medication) that in the past for other patients has led to the target medical outcome is presented. Optionally, the target medical outcome for the target patient is determined by correlating the respective patient parameter and healthcare provider interaction journeys to aggregated medical data collected from multiple subjects, and extracting the target medical outcome from the aggregated data. Optionally, an alert may be generated, for example, in exceptional circumstances. In another example, the computed correlations are between a workflow pattern represented as the interaction journey and one or more patient parameters, for example, a urinary catheter is changed on a regular basis at a defined frequency (e.g., obtained from digital, analog, or voice nursing records) for a patient having a certain diagnosis stored in the EMR and/or when physician orders to change the catheter still stand and have not been fulfilled. The correlations may be computed for the current patient based on the current frequency of changing the urinary catheter, and/or for other patients based on the frequency of changing the urinary catheter for other patients (optionally similar patients, for example, similar diagnosis). The model may output an alert when the urinary catheter has not been changed according to the learned frequency and when the certain diagnosis and/or physician orders still stand.

Optionally, the model is trained according to computed correlations with one or more other data sources as described herein, for example, sound and/or images (e.g., video) of the interaction of the healthcare provider with the patient and/or other healthcare providers, the identity profile of the healthcare provider, and/or aggregated medical data. Optionally, the model is trained according to computed correlations between the one or more other data sources and the interaction journeys of the plurality of sample healthcare providers and/or the patient parameter of the sample patients.

The correlations may be dynamically computed for dynamic updating of the model, for example, as new interaction journeys and new patient parameters are available.

Optionally, the model is trained by training sub-components at each of multiple different entities, for example, hospitals, clinical, health maintenance organizations (HMO), and nursing homes. Such different entities and organizations may not have a cooperation agreement, may compete with each other, and/or may not want their data to be transmitted outside the organizing. A central model may be created by aggregating the sub-components and creating a new, more general aggregated model. Training sub-components at different entities helps ensure privacy of the data used to train the sub-component, since the data does not leave the entity, the data is not exposed over the network and/or at other sites and/or to malicious intent. The data may be kept secure at each site. The aggregated model learned from some or all the models of the sub-components benefits from and encapsulates the data and interaction journeys at all of the participating organizations and enables a better model.

As used herein, the term model may refer to one or multiple models, for example, artificial intelligence code and/or machine learning code and/or adaptive system and/or statistical classifiers. The model may include multiple components, for example, a statistical classifier and/or other code. For example, multiple components may be trained, which may process data in parallel and/or as a pipeline. For example, output of one type of model (e.g., from intermediate layers of a neural network) is fed as input into another type of model. For example, a first component of the model is trained to detect the correlations, and a second component which receives as input the output of the second component is trained to output the adjustment to the UI. Exemplary models may include one or more statistical classifiers, one or more neural networks of various architectures (e.g., artificial, deep, convolutional, fully connected), support vector machine (SVM), logistic regression, k-nearest neighbor, decision trees, other implementations described herein, generative adversarial networks (GANs), and combinations of the aforementioned.

The distinguished characteristics between a certain number of data sets from contextual data and/or point data that are applied to the given treatment and/or cases that are studied may be identified. The number of nodes that are checked may be dependent on the case and/or varied from case to case (also referred to hereinafter as the “distinguished data set”).

When the data is definable and/or labeled, the model may be implemented as supervised, semi-supervised, unsupervised, and/or other technique to perform the analysis. When the data lacks labels, the model may be implemented as a semi-supervised, unsupervised, and/or other technique to perform the analysis, or labels may be added and then labeled data techniques may be used. Calibration to different parameters of the data set may be performed, in order to distinguish the given amount of the participating sets. Thus, the model attempts to learn the parameters that most closely identify and/or distinguish the given amount of classes in the system.

The model may be implemented based on optimization methods, for example but not limited to, are the method of gradient descent, random forest, K means, least squares, etc. These processes tune the parameters to find the most optimal models and/or other appropriate analysis technique of the two or more classes depending on the study cases. Other implementations (e.g., strategies and/or methods) may be used in order to provide the overall insight and/or detailed treatment overview. The model may use regression, for example, but not limited to, in order to understand how typically the data's value of one dependent variable changes while other independent variable vary or stay fixed. Those statistical strategies may be part, but not limited to, or in combination with other techniques or methods, be part of the basis of the analysis.

Exemplary implementations of the model include, for example, supervised, unsupervised, semi-supervised, kernel methods, and/or other machine learning strategies. Optimization, regression, statistics, and/or other strategies may be used to achieve advanced analysis. Each process may be used as appropriate for a particular situation.

At 152, the interaction journey of the user with one or more medical devices is received. The interaction journey may be implemented as a data structure that stores interactions of the user with the one or more medical devices and/or images and/or parameters, for example, which actions the user performed on the medical device, such as which buttons the user pressed on the medical device, which data the user viewed (i.e., what data was presented by the medical device and/or outputted by the medical device during the interaction), and/or data the user entered into the medical device (e.g., setting and/or adjusting parameters of the device, entering patient data into the EMR) and/or the action of the user marking a UI and/or which data the user marked on the UI (e.g., marking an anatomical feature, tumor, and/or abnormality, etc. on an image on a touch screen, such as by a circle around the anatomical feature).

The interaction journey may capture the sequence of interactions of the user with the medical device(s), for example, the following are performed sequentially: patient EMR currently presented on a touch screen, user touches a new patient diagnosis, user opens a PACS window, user selects an x-ray for viewing, and user changes a dose of a medication.

The interaction journey is optionally of multiple medical devices, for example, capturing interaction with a GUI presented on a display, with a patient ventilator, with a patient blood pressure sensor, with an EMR, and with a PACS server. The sequence of interactions may be of interactions with the multiple medical devices, for example, the order of actions performed with the multiple medical devices.

The medical devices may, for example, store data of the patient (e.g., EMR, medical images), monitor the target patient (e.g., ICP sensor, blood pressure sensor), and/or treat the patient (e.g., ventilator).

The interaction journey may be stored, for example, as a map, a graph, code, text, number, and/or other formats. The interaction journey may define, for example, selections made by the user, input provided by the user, and/or adjustments to the medical device made by the user.

The interaction journey may capture the interactions of the healthcare provider with multiple medical devices, for example, accessing the EMR to obtain a certain blood test result, then checking a medical image, then setting the ventilator to certain values, then adjusting the pulse oximeter, selecting which system (e.g., PACS, medication order system) to present on a large or standard-sized screen, touching values of blood test results on a touch screen, writing notes on a digital screen with a special pen, entering patient treatment orders into a digital patient chart, and adjusting a presentation of data on a screen (e.g., zoom-in, open app, download app, perform action in app, minimize window, open minimized window, highlight data, and arrange order of data on the screen).

Exemplary medical devices include: electronic health information system, electronic medical or health record, medical imaging system, medical image, patient monitor, anesthesiology monitor, physiological sensor or monitor, intracranial pressure sensor, cerebral perfusion pressure sensor, arterial line, respiration device, blood pressure sensor, temperature sensor, and pulse oximeter.

The interaction journey may be computed, for example, based on one or more of: analysis of images captured by a camera of the healthcare provider interacting with the respective medical device, analysis of interaction of the healthcare provider with a dedicated display of the respective medical device, analysis of interaction of the healthcare provider with a generic display of a computing device connected to the respective medical device, analysis of interaction of the healthcare provider with buttons of the respective medical device, interface of the medical device outputting data indicative of the interaction, a microphone capturing any such interaction, analysis of interaction of caregiver with the computing device, interaction with a microphone (e.g., speaking into a voice recognition system, calling for a certain physician using a public service speaker), pen interactions, camera images and/or video, selecting audio for playing on speakers, use of headphones, use of VR glasses, capturing patient events using camera, microphone, video, etc., and combinations of the aforementioned.

Optionally, the interaction journey includes and/or is of the healthcare provider with a UI presented on a display. The UI may be a GUI. The GUI may be presented on a display, including presenting the multiple patient parameters that are monitored as described herein. Optionally, the interaction journey is based on at least one of: touch interaction with a touch screen presenting the UI, pen capable of writing on the touch screen, dial or wheel placed on a screen presenting the UI, a keyboard, a port receiving data from another user interface device or the network, and a camera capturing images of the interaction with the UI

Exemplary interfaces that may be used for monitoring the interaction journey include: touch screen, camera, keyboard, pen, dial, microphone, speaker, headphones, virtual reality (VR) glasses, smart glasses, and augmented reality (AR) headset.

Exemplary interactions performed by the user that are included in the interaction journey include, for example: zoom-in on a certain monitored patient parameter, selection of a certain monitored patient parameter for presentation in the UI, removal of a certain monitored patient parameter from the UI, relative positioning between two or more monitored patient parameters in the UI, marking a certain monitored patient parameter with an indication of importance, entering data, entering a diagnosis, entering orders for treatment, camera images of caregiver treating patient, camera images of caregiver activities (such as washing hands), camera images of patient events (such as cough, seizure, sneeze, fall, or other event), camera images of patient activities, microphone sound captures of patient events, microphone sound captures of activities, microphone sound capture of caregiver events, activities, or interaction with patient, other caregiver, or others (such as family members), interactions of caregiver or other with pen, wheel, dial, keyboard, mouse, or similar input device, and the like.

The interaction journey may include use of different information sources, for example: did a physician check a certain parameter, measurement, image, etc. in a certain scenario. By way of another example: did a physician correct a certain parameter recorded by an automated system. By way of another example: did a physician zoom or focus on a certain parameter or a certain part of an image.

The interaction journey may include a focus on different information sources and/or actions. For example, when a physician checks a CT scan, does he or she also change the dose of a certain medication.

At 154, one or more patient parameters are received. The one or more patient parameters may be monitored. The patient parameters are of the target patient.

The patient parameters may be presented on a UI, optionally GUI, presented on a display for which the interaction journey is computed. For example, the patient parameters are presented on a large touch screen beside the bed of the patient. In another example, the patient parameters are displayed on a tablet carried by the caregiver. In another example, the patient parameters are displayed on a centralized display, such as a display at the central nurses' station, that displays the information of multiple patients. The interaction journey is computed for the interactions with the healthcare provider using the UI presenting the patient parameters, for example, which data the healthcare provider selected to look at, medication ordered entered via the UI, and other examples as described herein.

Optionally, one or more of the patient parameter is indicative of a current medical state relative to a target medical outcome of the target patient. The model outputs the adjustment to the UI for increasing likelihood of reaching the target medical outcome. For example, the patient parameter is blood pressure, and the target medical outcome is a target blood pressure range. In another example, the patient parameter is a certain medication dose, and the target medical outcome is termination of the medication. In another example, the patient parameter is that the patient is currently in the ICU, and the target medical outcome is discharge or transfer to the internal medicine ward.

Optionally, one or more of the patient parameters are outputted by the medical device(s) for which the interaction journey is monitored. For example, the interaction journey of the healthcare provider with a ventilator is monitored, and values measured by the ventilator are the patient parameters.

Optionally, the patient parameters include output of physiological sensors that each measure a respective physiological parameter of the patient, for example: medical images, blood pressure measurement devices, arterial line, respiration devices, resuscitation devices, monitors, body temperature measurement devices, patient monitor(s), intracranial pressure sensor, Cerebral perfusion pressure sensor, or other common medical devices in the patient room, hospital, or medical clinic or office.

Optionally, the patient parameters are obtained from data sources (optionally non-physiological data sources) that each store a respective parameter of the patient (optionally non-physiological parameter), for example: patient demographics, identity profile of healthcare provider team members, history of the present illness, prior medical history, prior treatments, previously scheduled appointments, future scheduled appointments, treatment facilities where the target patient was treated. It is noted that one or more of the patient parameters may alternatively or additionally be represented in the monitored interaction journey, for example, the nurse may schedule a future appointment using a UI of a computer, which is captured as in the interaction journey.

Optionally, the patient parameter is obtained from one or more care process data sources that each store a respective care process parameter of the patient. Examples of care process data sources include, for example: which parameters each caregiver referred to, identity of treating caregiver, position of treating caregiver, expertise of treating caregiver, decisions of processes chosen by treating caregiver, actions of treating caregiver within the system, requests and referrals of treating caregiver; clicks, searches, time spent on each parameter, system overrides, corrections, re-checks, focuses of treating caregiver. Care process data sources and care process parameters may overlap with patient parameters and/or with interaction journeys. For example, an action performed by the user captured in the interactive journey may alternatively or additionally be represented as the care process data source and/or patient parameter. For example, care process data source and/or patient parameter may alternatively or additionally be represented as an action performed by the user captured in the interactive journey.

Patient parameters may be visualized, and/or collected. Every data element may be collected digitally (as oppose to manually) with digitized manuals which are included, but not necessarily limited to, ICU charts, blood pressure chart, temperatures chart, resuscitation charts, files, pages, and/or measurement paper system that may be built in and/or which replaces the hand written manuals and/or is located in the personal system and/or data storage of the individual target patient.

At 156, additional data is received. The additional data may be monitored.

The additional data may be obtained, for example, manually entered by a user, automatically obtained from the patient electronic medical record, and/or automatically obtained by accessing a remote server (e.g., hospital administration server, healthcare insurance server, government server).

Exemplary additional data include one or more of:

Sound and/or images (e.g. video) of an interaction of the healthcare provider with the patient and/or other healthcare providers, for example, discussions held by the healthcare team at the patient bedside, and/or a physical examination of the patient performed by the healthcare provider, and/or discussion between the healthcare provider and patient.

Referral to another healthcare provider, for example, calling a specialist for evaluation of the patient, transfer to another ward, transfer between on-shift teams, and/or transfer for care by another physician.

An identity profile of the healthcare provider. The identity profile may include, for example: position of the healthcare provider (e.g., nurse, medical student, resident, staff physician) medical training rank, medical training type, external reviews or ranks such as those by research and medical publications, medical publication citations, medical specialty, demographic data, previous experience in performing medical procedures, medical skills, and success in treating other patients.

Identity of the patient, demographic data, for example, obtained from the patient electronic medical record, manually entered by a user, obtained by accessing a remote server (e.g., government record server).

Identity of the user (e.g., caregivers) and/or credentials of the user (e.g., user profile): nurses, head nurses, residents, physicians, senior physicians, professors, department heads, etc. Obtained, for example, by a record linked to a login entered by the user, manually entered by a user, obtained by accessing a remote server (e.g., hospital administration server).

Prior medical treatments, and optionally results, for example, improvement, no change, or worsening of condition.

Caregivers' train of thoughts, provided, for example, by a microphone that records the discussion amongst the healthcare team treating the patient and code that converts the audio to text.

Experience, pedigree, and/or specialty of the given user (e.g., physicians or other caregivers), for example: a certain treatment decided on by a nurse, nurse practitioner, resident, senior physician, department head, etc. By way of another example only: was a certain treatment performed by a nurse, nurse practitioner, resident, senior physician, department head, etc.

Aggregated medical data into the model, for example: medical research publication, and medical treatment guideline.

Patient profile data. The patient profile data may include one or more of the patient parameters. For example, demographics, previous medical history, current medical diagnosis, and currently prescribed medications. The patient profile data may be used by the model for computing correlations for patients having similar patient profiles, for example, a correlation value computed between the patient profiles of the current patient and other patients above a threshold.

Prior treatment given for the medical condition of the patient.

Other prior and/or current caregivers treating the patient, i.e., concurrent, and/or other caregivers.

At 158, the interaction journey and/or one or more of the patient parameters and/or the other data is fed into the model.

Optionally, the interaction journey and/or one or more of the patient parameters and/or the other data are fed into a centralized model. Alternatively or additionally, the interaction journey and/or one or more of the patient parameters and/or the other data are distributed and fed to multiple sub-components of the model for example, hosted at different processing nodes. Sub-nodes may reside at multiple different entities, for example, hospitals, clinics, health maintenance organizations (HMO), skilled nursing facilities, and/or nursing homes. Each sub-component is trained (optionally locally trained) according to the interaction journeys and/or patient parameters and/or other data obtained from the respective local facility. Each sub-component is trained according to unique computed correlations between interaction journeys of unique healthcare providers with respective medical devices and the monitored patient parameters of unique sample patients and/or unique other data. Training sub-components at different facilities helps ensure privacy of the data used to train the sub-component, since the data does not leave the entity, the data is not exposed over the network and/or at other sites and/or to malicious intent. The data may be kept secure at each site. Outputs of multiple sub-components may be aggregated into a single output of an adaptation to the UI, for example, a majority vote, and/or executing all different types of adaptations.

A central model may be created by aggregating the sub-components.

At 160, the model processes the fed interaction journey and/or patient parameter(s) and/or additional data. The model may compute one or more correlations.

Optionally, the patient profile of the current patient is used to find one or more other patients having similar patient profiles (e.g., computing a correlation value between the patient profiles, where the correlation value is above a threshold). The model may compute the correlations for the matched patient profiles.

The correlations may be selected, for example, according to statistical significance (e.g., statistical value above a threshold), according to predefined correlations, according to correlations found during training, according to correlations defined by a user as significant during training, and/or other processes.

“Point data” (i.e., patient medical data) and “contextual data” (e.g., the interaction journey and/or other data) may be correlated and/or combined using computerized mathematical and digital processes (i.e., the model) to learn, analyze, and/or understand the condition of the patient, and how and where to treat the patient in a given situation.

Correlations may be multi-dimensional, between two, three or more parameters, for example, second, third, and higher order analysis. By way of example: correlations and any other relations of the “point data” and “contextual data” described herein.

Exemplary correlations may be based on the interaction journey, and one or more of the other data described herein, which may be medical data elements presented on the UI and/or one or more other medical data elements.

At 162, an adaptation to the UI is outputted by the model. The UI may be, for example, presented on a screen, presented on VR glasses, presented on an augmented reality (AR) headset, played as audio on speakers, and/or other implementations. The UI may be a GUI.

Optionally, the model outputs instructions for adaptation to the UI. Alternatively, the model outputs an indication of the adaptation. The instructions may be generated by another code process that receives the indication of the adaptation as input. Instructions for adapting the UI may be provided, for example, using a code where each value of the code outputted by the model maps to a certain adaptation, code for adapting the UI, and/or other implementations.

Optionally, the adaptation to the UI outputted by the model is computed for increasing likelihood of the current medical state reaching the target medical outcome, as described herein. For example, when the current medical state is a current value of the monitored patient parameter, and the target medical outcome is a target value or target range or target threshold, the adaptation to the UI is selected to increase likelihood of reaching the target value.

Exemplary adaptations to the UI include: zoom-in on a certain monitored patient parameter, marking a certain monitored patient parameter to attract attention of the healthcare provider, selection of a certain monitored patient parameter that is not presented in the UI for presentation in the UI, removal of a certain monitored patient parameter from the UI, relative positioning between two or more monitored patient parameters in the UI, presenting a message indicative of a recommendation for manual adaptation of the UI, presenting at least one aggregated medical data collected from multiple subjects, presenting a suggested diagnoses in the UI, presenting a suggested treatment plan in the UI, presenting one or more parameter of a customizable period of time in the UI, playing an audio message on speakers, presenting an augmented reality image on an augmented reality headset, presenting a virtual reality image on virtual reality glasses, and presenting data of another or similar patient or medical case.

Optionally, the adaptation to the UI includes an alarm that is generated when one or more correlations are according to a requirement. The alarm may be, for example, a text message presented on the UI, a marking of a value indicative of the alarm, a beep or other sound, and/or presenting of a certain patient parameter value for extra attention and/or close monitoring. The requirement may be automatically learned by the model and/or manually defined by a user. The requirement may be defined as a threshold, range, and/or set of rules. The requirement may be defined as associated with a likelihood of a target medical outcome, which may be desirable or to be avoided. For example, when the correlation is associated with increased risk of heart attack, the generated alert is a text message suggesting to monitor the patient for a heart attack. In another example, when the correlation is associated with increased likelihood of discharge over the next 8 hours, the generated alert is a text message suggesting to evaluate the patient for discharge.

At 164, an adapted UI is created by implementing the adaptation (i.e., instructions for adaptation) outputted by the model.

At 166, the model may be updated based on the current iteration of the interaction journey and/or patient parameters and/or other data.

Alternatively or additionally, the model is updated based on an update received from an external entity, for example, a facility other than the one which employs the current user of the UI. The update may include a newly discovered correlation and/or newly discovered adaptation. The update may be distributed for updating sub-components of the model located at each facility, and/or a central model.

At 168, one or more features described with reference to 150-166 are iterated. The iterations may be performed, for example, for continuously (and/or per event) monitoring the interaction journey and new patient parameters and/or new other data, and dynamically updating the UI based on real time outputs of the model. The iterations may be performed, for example, by monitoring the interaction journey of the user interacting with the adapted UI (created by implementing the adaptations outputted by the model for the current UI), feeding the interaction journey with the adapted UI into the model to output another adaptation to the UI, and updating the previously adapted UI according to the new adaptation. In this manner, the interaction of the user with the dynamically adapted UI created based on the model is monitored and adjusted.

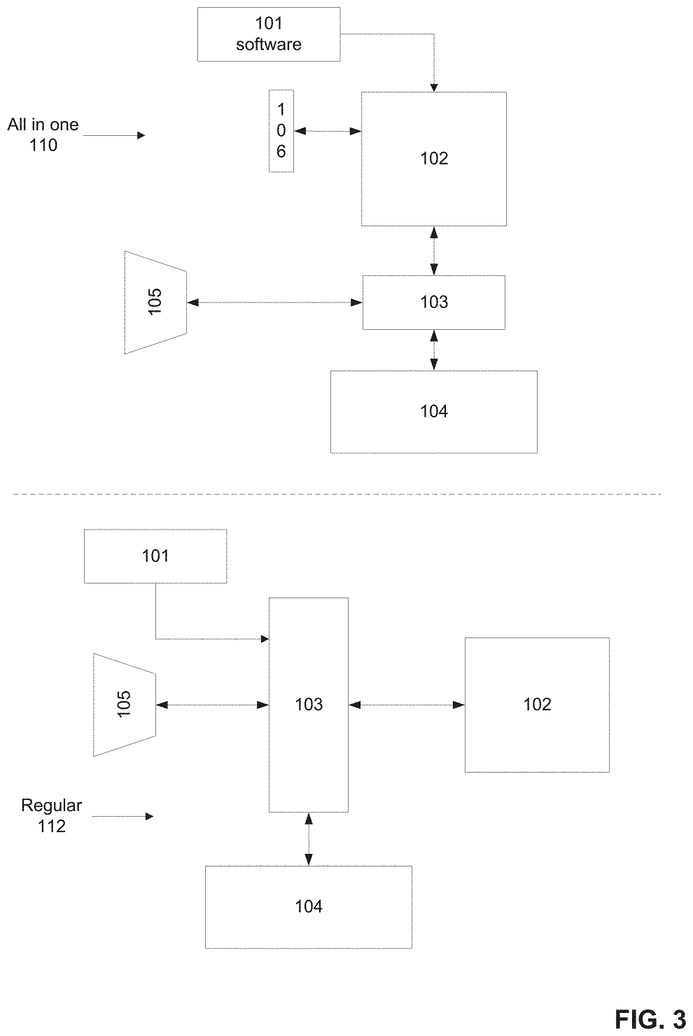

The systems and/or methods and/or UI described with reference to FIGS. 3-15 may be implemented based on, combined with, and/or substituted with, one or more features and/or components of the method described with reference to FIG. 1, and/or system 200 described with reference to FIG. 2.

Reference is now made to FIG. 3, which is a block diagram of another exemplary system for adapting a UI for presenting medical data for guiding treatment of a patient, in accordance with some embodiments of the present invention. Schematic 110 denotes an all-in-one implementation, for example, a standalone large touch screen that presents patient data thereon, and with which that user may interacted. The model may be locally executed by a processor of the touch screen, and/or the touch screen may be connected to the model execution on a network node via a network connection 105. Schematic 112 denotes a standard implementation, for example, where the model is centrally executing on a node and different devices are connected to the node. Schematics 110 and 112 may use common components, for example, 101 denoting code that when executed implements one or more of the features of the methods described herein, and 102, 103, 104, and 106 which denote exemplary input and/or output components, and 105 which denotes an exemplary communication component. Exemplary input components include one or more of: pen, keyboard, mouse, dial, wheel, touchscreen, camera, microphone, VR, AR. Exemplary output components include one or more of: screen, speaker, VR, AR, headphone, and headset.

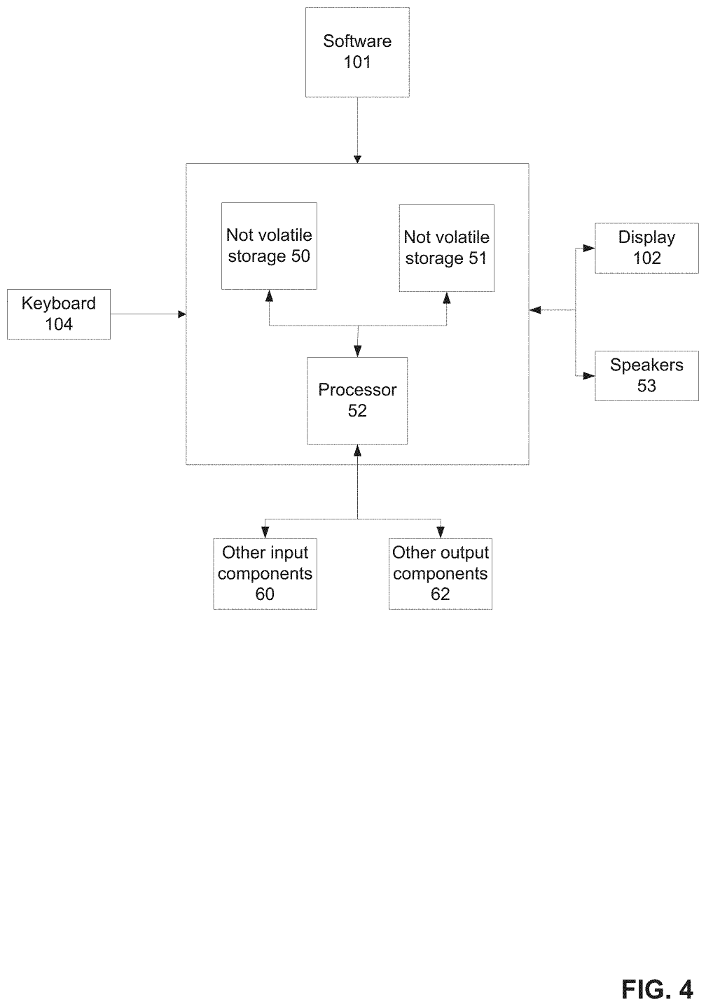

Reference is now made to FIG. 4, which is a block diagram of yet another exemplary system for adapting a UI for presenting medical data for guiding treatment of a patient, in accordance with some embodiments of the present invention. Exemplary components include: 101 denoting code that when executed implements one or more of the features of the methods described herein, keyboard 104, a non-volatile storage 50, a volatile storage 51, a processor 52, and display 102, and speakers 53. The system described with reference to FIG. 4 may include additional input components 60 and/or output components 62, for example, as described herein, for example, pen, keyboard, mouse, dial, wheel, touchscreen, camera, microphone, VR, AR, screen, speaker, headphone, and headset.

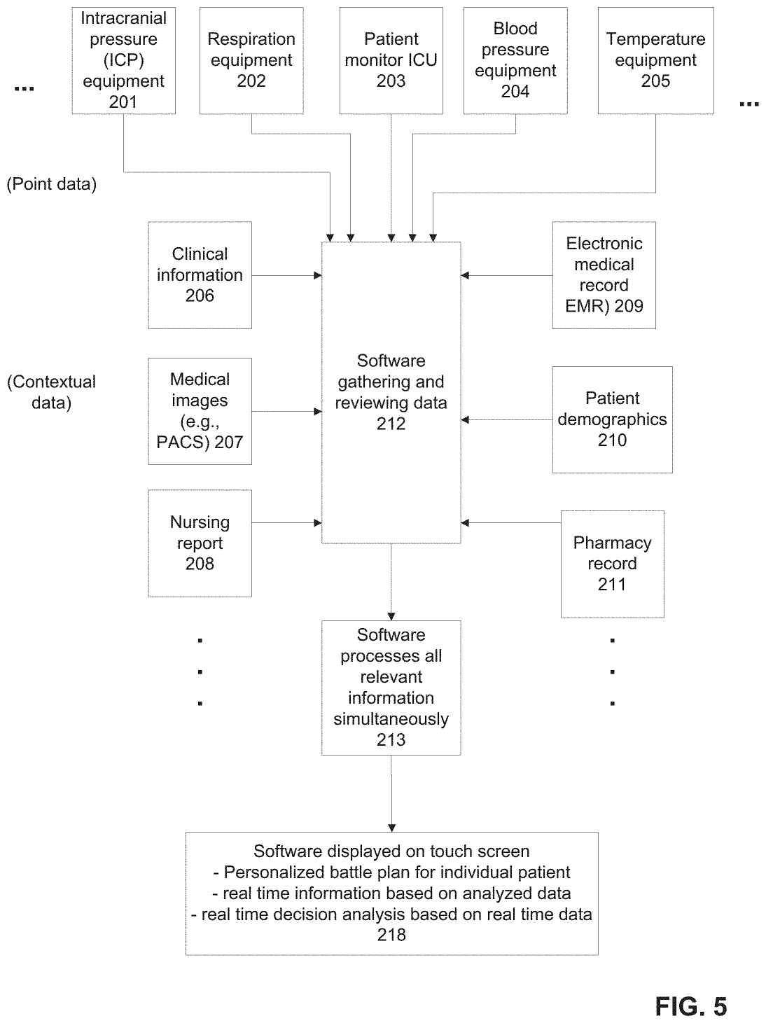

Reference is now made to FIG. 5, which is a dataflow diagram of an exemplary dataflow for correlating patient medical data and other data, in accordance with some embodiments of the present invention. Components 201-205 denote patient specific physiological measurement medical data (i.e., point data), for example, ICP device 201, respiration device 202, ICU patient monitor 203, blood pressure device 204 and temperature device 205. Components 206-211 denote other medical data of the patient, and/or indirectly related data of the patient (e.g., contextual data), for example, clinical information 206, medical images 207, nursing reports 208, electronic medical record 209, patient demographics 210, and pharmacy records 211. It is noted that components 201-211 are not necessarily limiting examples. Additional components may be added, components may be removed, and/or components may be replaced with other components. At 212, data from components 201-205 and components 206-211 is obtained, for example, by the computing device of FIG. 2. At 213, the data is processed and/or analyzed, for example, by the computing device as described herein. At 218, the UI is dynamically updated, as described herein, for example, with a personalized treatment plan for the patient, real time information based on the analyzed data, and/or real time decision analysis.

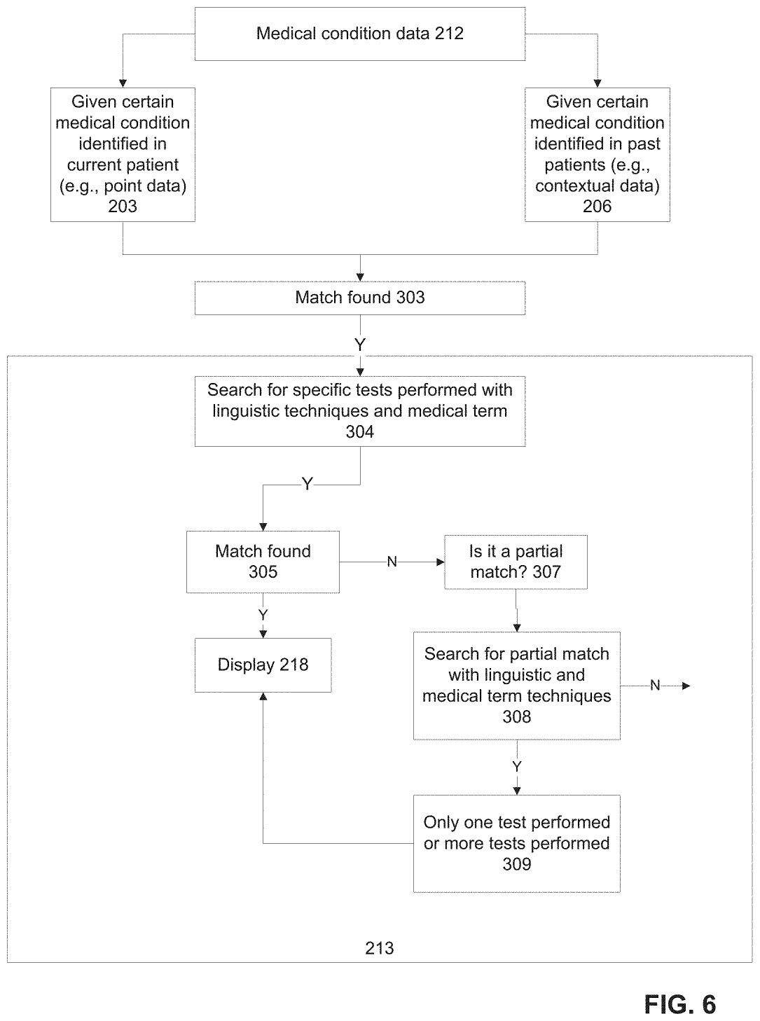

Reference is now made to FIG. 6, which is another dataflow diagram of an exemplary dataflow for correlating patient medical data and other data, in accordance with some embodiments of the present invention. Features of the method of FIG. 6 may be based on, and/or combined with, and/or substituted for, features of the methods of FIG. 5. At 212, medical condition data is obtained. At 203, the medical condition is identified in the target patient (i.e., point data). At 206, the medical condition is identified in one or more other patients based on the EMR (i.e., contextual data). At 303, a match is found between the current patient and one or more other patients. At 304 (of 213), medical tests and/or treatments performed for the matched patient are searched. At 305, a match is found. At 218, the test is presented in the UI. Alternatively, at 307, when no match is found, at 308 a partial search is performed. At 309, the partial match is found and presented at 218.

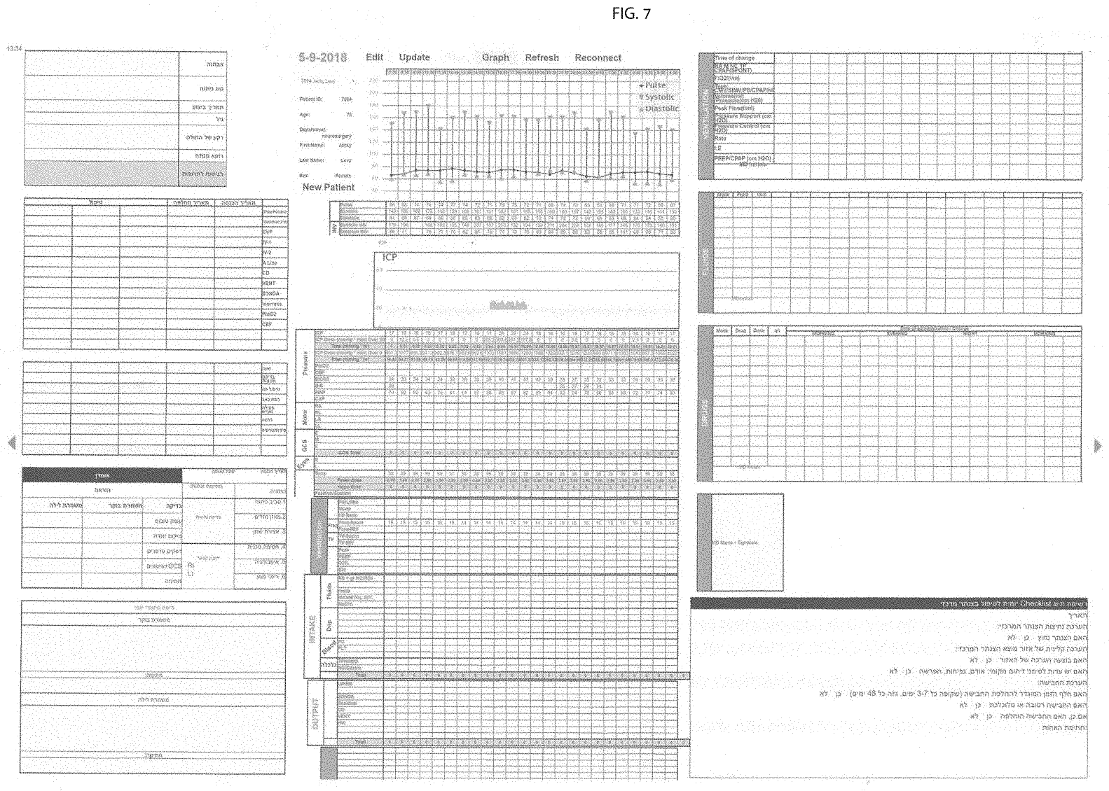

Reference is now made to FIG. 7, which is a schematic of a UI presenting patient parameters for a monitored patient, in accordance with some embodiments of the present invention. The data may be automatically collected, optionally in real time, and optionally digitized. The existing data collection methods that are done manually and/or automatically uploaded to the institution's computer system may be replaced and/or augmented. Data may be fluently collected in real time and/or every second or so (or other values), and/or much higher resolution (for example, but not limited to, 100-500 Hz) for wave data and/or transform the data and present the data as a graphical display, optionally in the UI which is adapted as described herein. For example, in contrast to the data that was collected every one to eight hours (or more) using traditional processes. The wave display enables the medical personnel to understand, plan, and/or develop a comprehensive battle plan tailored for the individual patient's special needs. This may be especially valuable and/or lifesaving to patients that are in critical conditions. Other important data, not used by the personnel in this critical time, may be stored for later and/or further analysis and follow ups, and research. Optionally, the interaction with the UI presenting the data is monitored as the interaction journey that is fed into the model. The output of the model may iteratively adjust the UI.

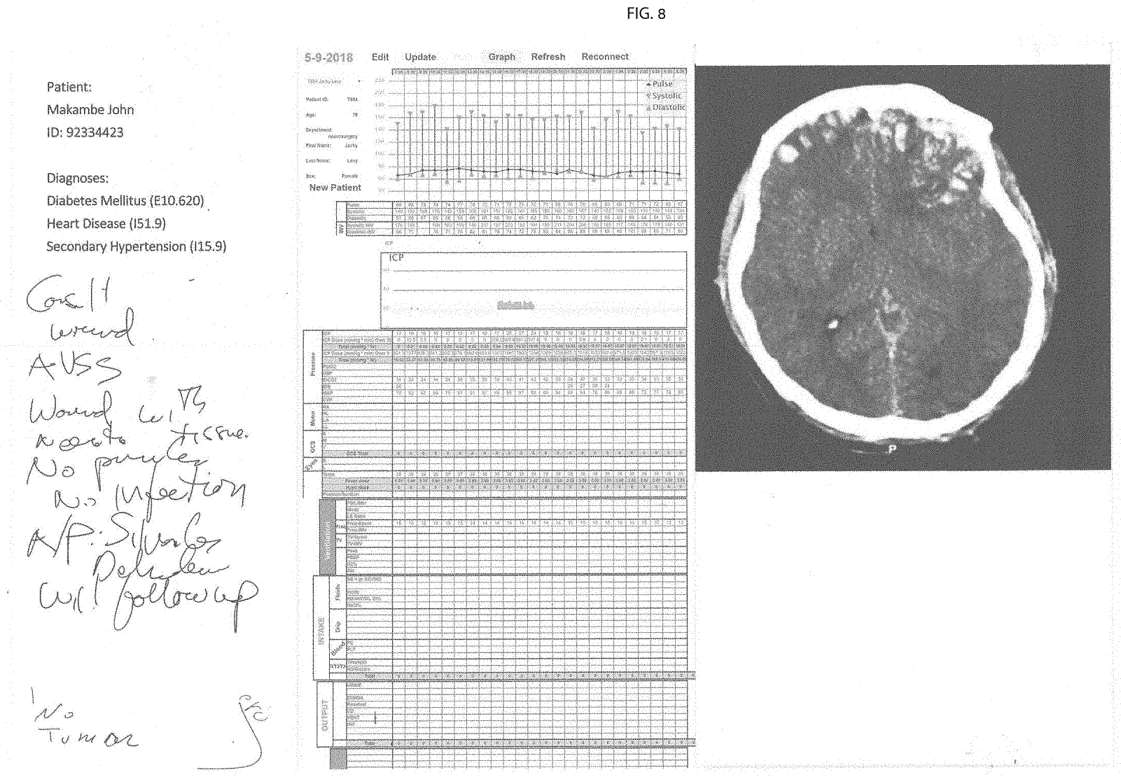

Reference is now made to FIG. 8, which is a schematic depicting an example of a UI implemented as a display presenting multiple patient parameters, in accordance with some embodiments of the present invention. The display may be a touch screen. The UI may be adjusted based on output of the model, as described herein. The interaction journey may be computed based on interaction of the healthcare provider with the display.

Optionally, the UI enables a screen split capabilities and/or display at the same time, for example, into two or three or more different views for analysis of patient data and/or analysis in front of the caregiver and/or patient. For illustration only and without necessarily limiting the endless possibilities of the screen capabilities, the screen may have a capability to display the patient's chart alongside a medical image and/or alongside a 3D medical image at the same time. This capability may enable the personnel to decide in real time on a personal treatment plan for the individual patient. Notes may be added and may be left on the screen for follow-ups and discussions, for example, using a pen like device designed for writing on the display. The interaction journey may include, for example, the writing with the pen like device on the display.

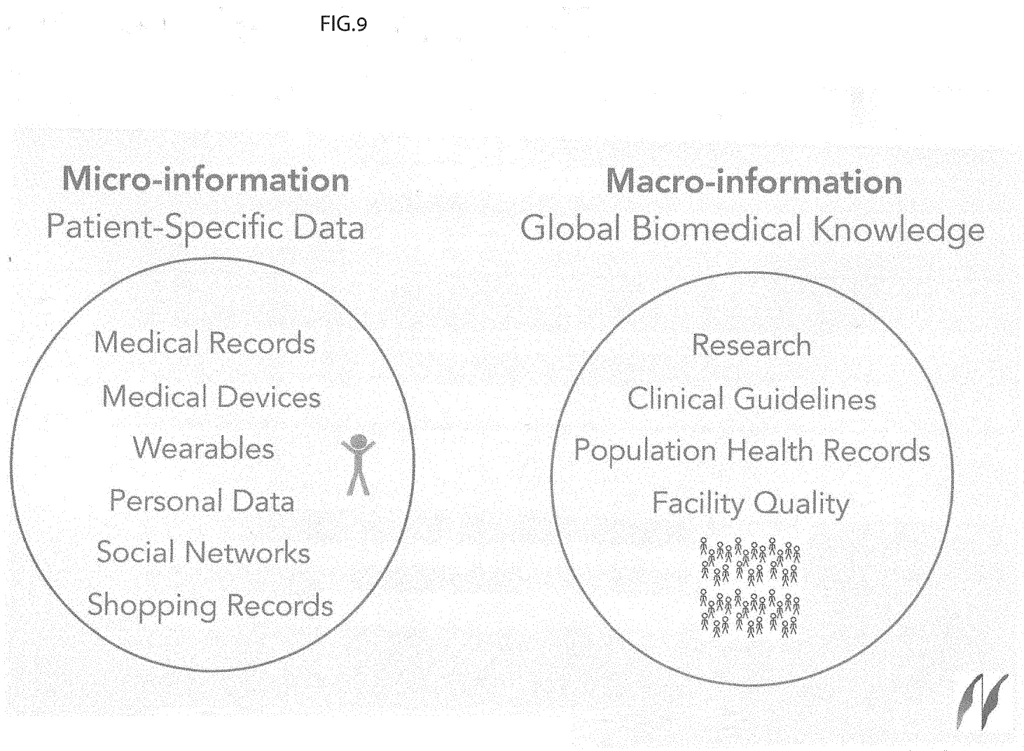

Reference is now made to FIG. 9, which is a schematic depicting exemplary data sources that stores and/or output data for computation of the patient parameters (labeled as micro-information), and exemplary aggregated data sources (labeled as macro-information), which are fed into the model with the interaction journey for outputting the adaptation of the UI, in accordance with some embodiments of the present invention. Patient parameters data sources (labeled as micro-information) are for each target patient, for example, Electronic Medical Records (EMRs), Electronic Health Records (EHRs), Wearables/Health apps, medical sensors, and genetic information. Aggregated data sources (labeled as macro-information) represent global knowledge of the biomedical community for multiple different patients (i.e., not patient specific), for example, previous treatment experiences, biomedical research, patient outcomes, and facility information such as facility ranking, facility quality, facility expertise, physicians who practice at the facility, etc.

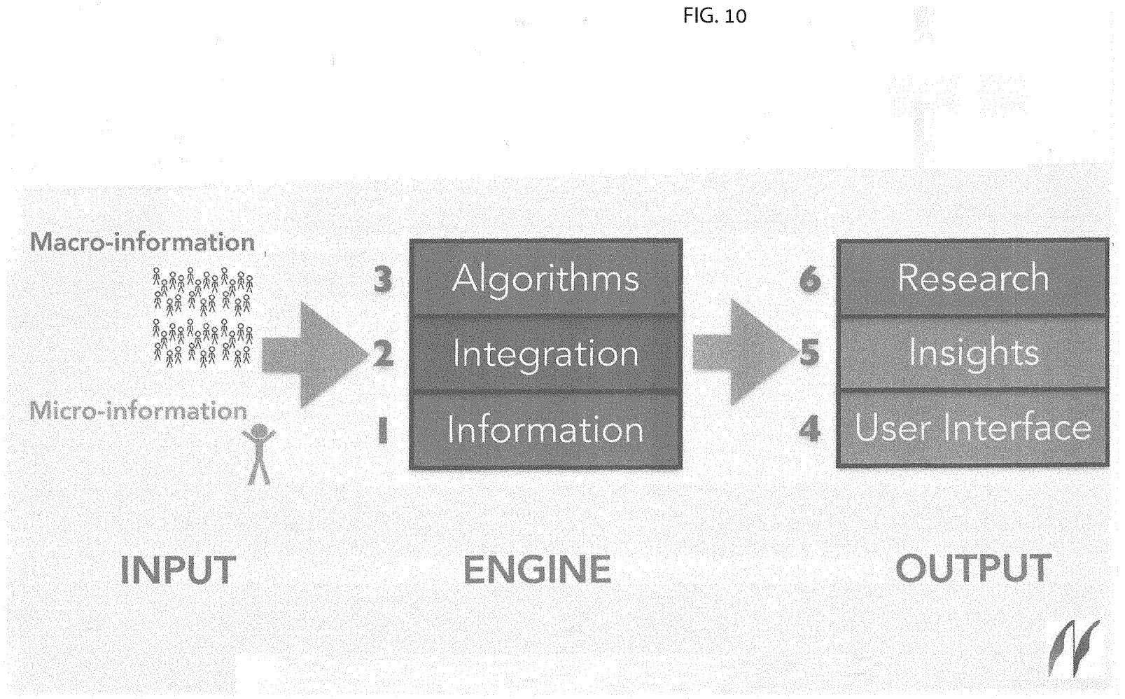

Reference is now made to FIG. 10, which is a high level data flow depicting the process of analyzing output of the model fed the interaction journey and patient parameters, in accordance with some embodiments of the present invention. The interaction journey, patient parameters (i.e., macro-information), and optionally micro-information are fed into the model (i.e., engine), which outputs instructions for adaptation of a UI. The output of the UI may be used, for example, or research and/or insights.

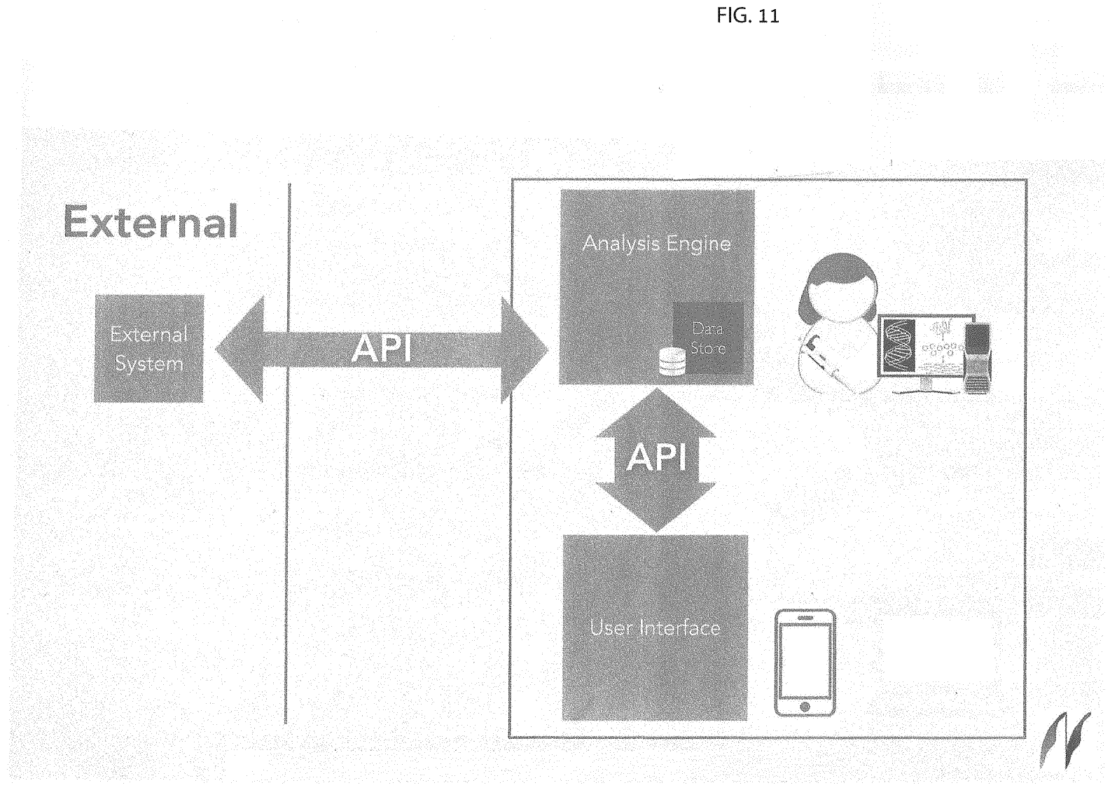

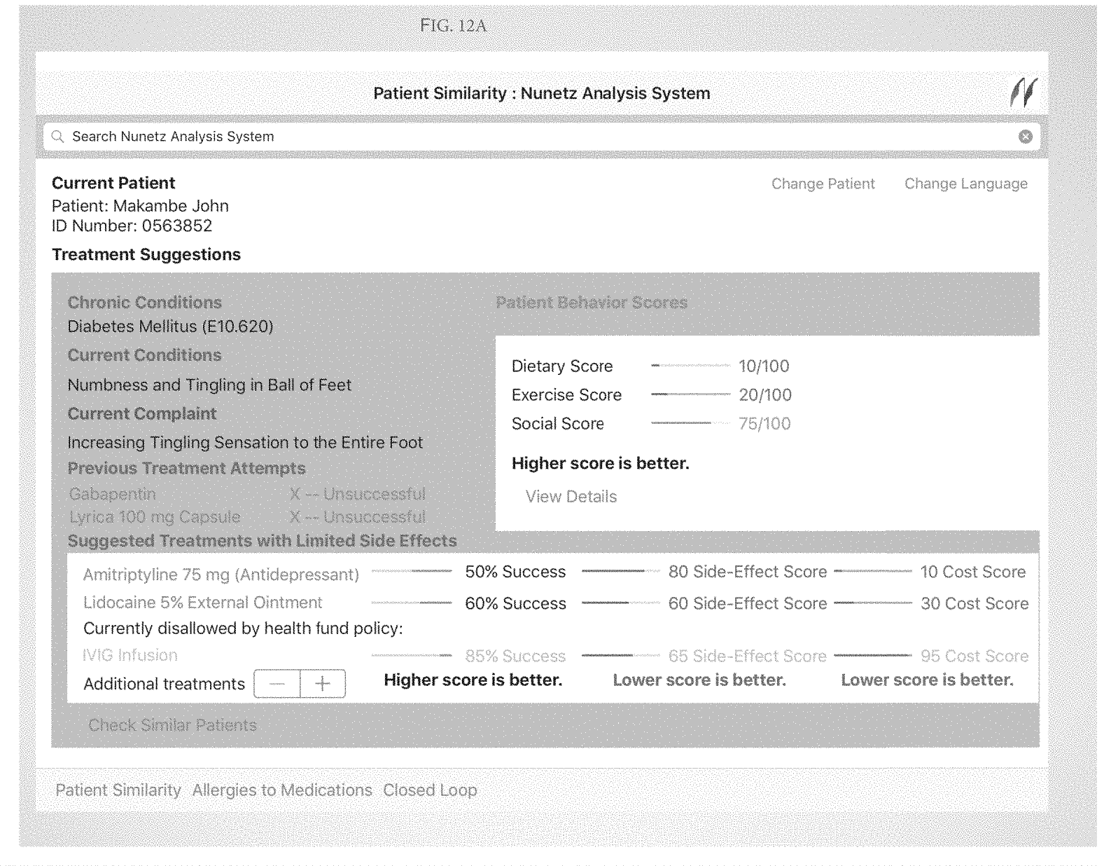

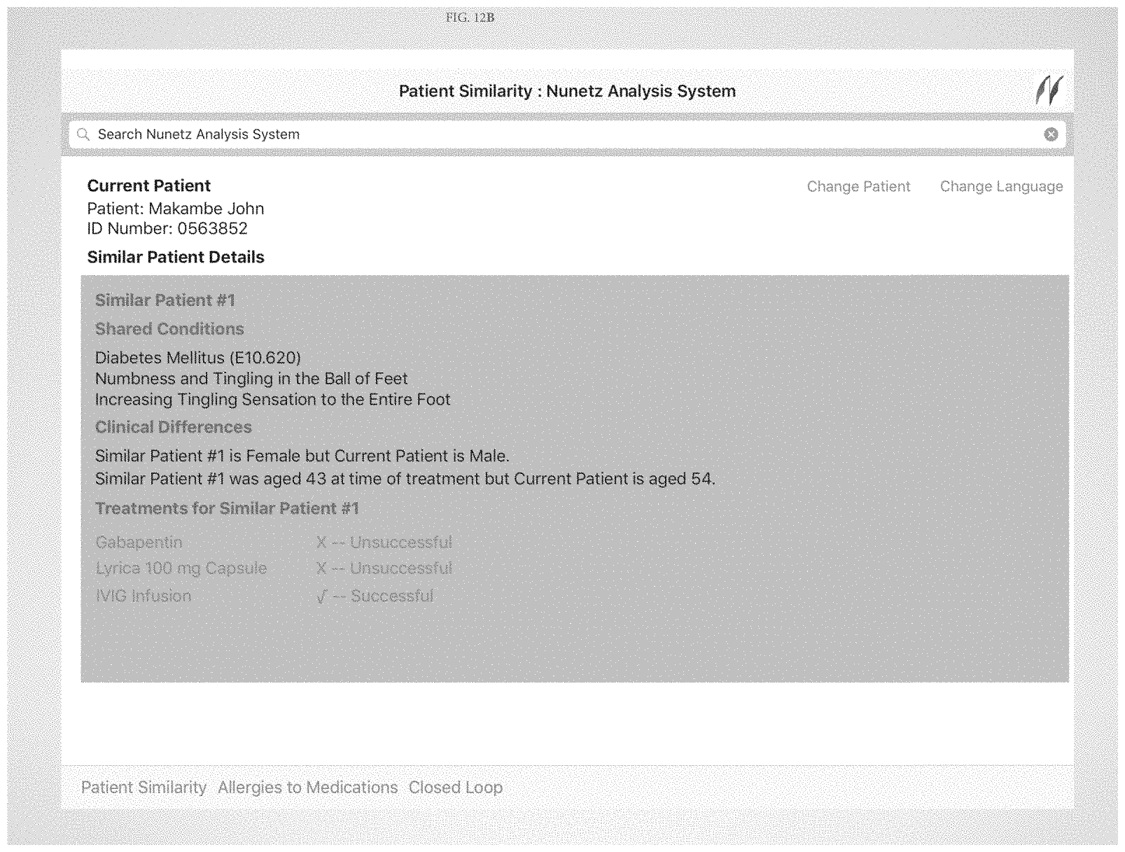

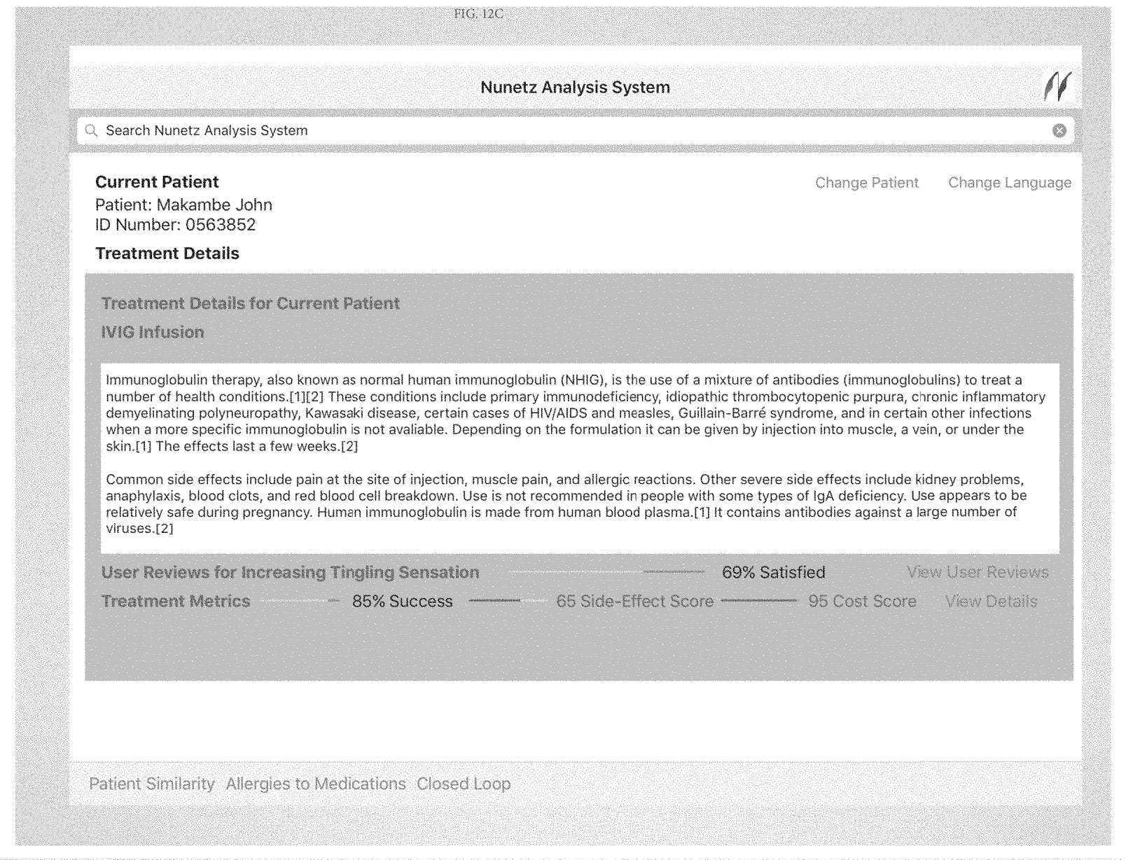

Reference is now made to FIG. 11, which is a schematic depicting an application system and/or application programming interface (API) for interacting with external entities, in accordance with some embodiments of the present invention. This feature may enable third parties such as other companies and/or users such as physicians, nurses, researchers and other caregivers to develop software and/or hardware to extend the model and/or use the data the model outputs. The applications may be displayed using the split screen system described herein. The APIs may be used, for example, to receive updates to the model created by third party entities, and/or to distribute the updates to multiple instances of the model, as described herein.

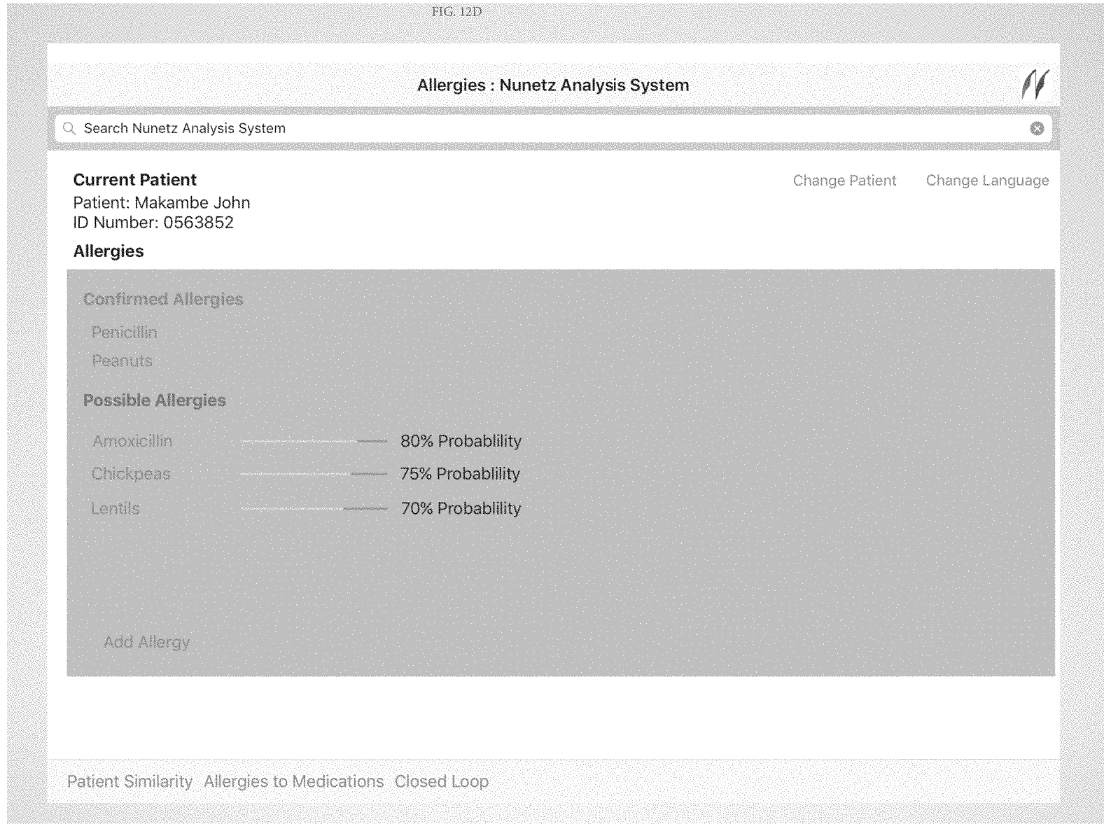

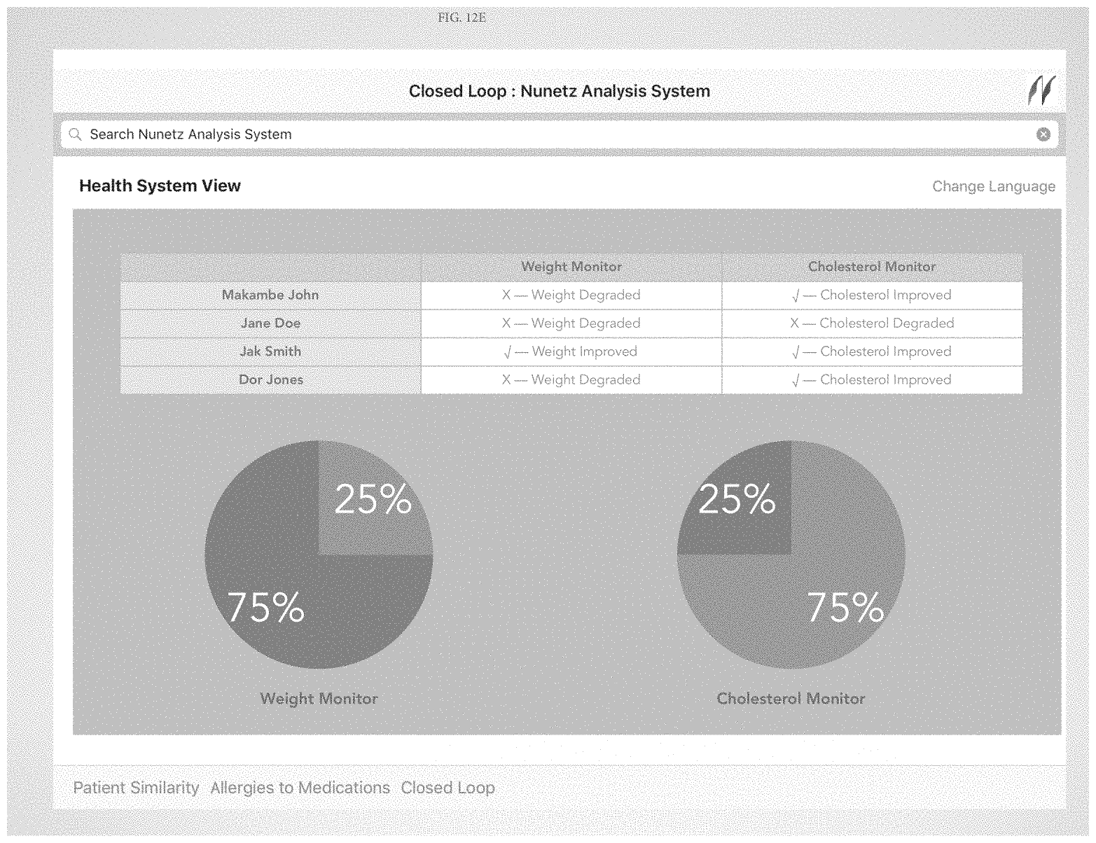

Reference is now made to FIGS. 12A-E, which are exemplary schematics of the UI adapted based on output of the model fed the interaction journey and patient parameter(s), in accordance with some embodiments of the present invention. FIGS. 12A-E may be dynamic updates of the UI based on a dynamic output of the model fed the dynamic interaction of the user with the UI and/or patient parameters and/or other data sources, as described herein.

In FIG. 12A, treatment suggestions are computed and presented based on output of the model. Optionally, scores and/or indications of success of previous treatment attempts for treatment of diabetes mellitus (e.g., unsuccessful treatment using gabapentin and lyrica, suggested treatments with limited side effects including amitryiptyline, lidocaine, and IVIG infusion) are outputted by the model and presented. For example, when the interaction journey is of a current user looking at the EMR of the patient to determine which treatments were used before and/or looking at records of patient diet, exercise and social score (e.g., computed using environment data and contextual data described herein), and when the patient's diagnosis of diabetes mellitus is stored in the EMR.

In FIG. 12B, a profile of a similar patient at least also diagnosed with diabetes mellitus is computed and presented based on output of the model. Differences between the target and similar patient are computed and presented based on output of the model. Indications of success and/or failure of treatments provided to the similar patient are outputted by the model and presented, i.e., the treatments of gabapentin and lyrica were unsuccessful for treating the matched similar patient but WIG infusion was successful. For example, when the interaction journey is of a current user searching the EMR for other similar patients, and/or looking at treatments of the similar patients, and optionally interaction journeys of other physicians looking for similar patients with a similar diagnosis that were successfully treated using a certain medication.

In FIG. 12C, an indication of satisfaction, success, and/or side-effect score is computed for the treatment which was found to be successful (i.e., IVIG infusion) for the target patient by the model and presented in the UI. For example, based on the interaction journey of the user looking at IVIG infusion treatment, metrics computed for other patients successfully treated with IVIG as stored in the EMR, and optionally interaction journeys of other physicians looking at successful treatments of other patients.

In FIG. 12D, an indication of probability of the patient being allergic to one or more substances is computed by the model and presented. For example, based on the interaction journey of other physicians that looked at allergies, and a history of the substances the patient is allergic to according to the EMR of the patient, when the interaction journey of the current user is looking at allergies and/or when the target patient is allergic to a substance.

In FIG. 12E, a summary and details of patients that lost weight (or gained weight) and have improved cholesterol (or degraded cholesterol) is outputted by the model and presented. For example, based on the interaction journey of other physicians that looked at weight and cholesterol values for multiple patients that were diagnosed with diabetes mellitus and/or for patients for which treatment for diabetes mellitus is being planned, for example, when the interaction journey of the current user is looking at multiple values of weight and/or cholesterol for the target patient over a time interval and/or when the user is planning treatment of the patient, and/or when the current patient is diagnosed with diabetes mellitus as stored in the EMR.

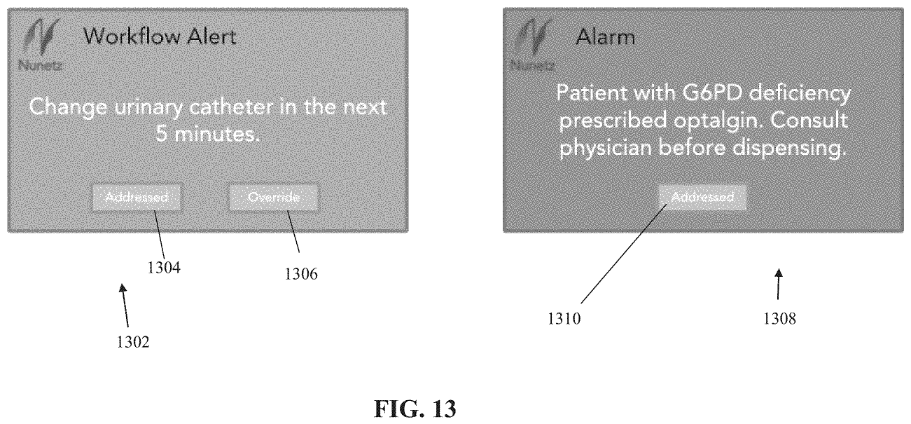

Reference is now made to FIG. 13, which is a schematic of an exemplary workflow alert 1302 and/or exemplary alarm 1302 outputted by the model, in accordance with some embodiments of the present invention. Workflow alert 1302 indicates an upcoming action that is to be performed on the patient, which is learned by the model. For example, the model learns that the urinary catheter should be changed in the next 5 minutes after learning the frequency at which urinary catheters are changed based on the interaction journey of the current healthcare provider and/or other healthcare providers. Buttons may be provided indicating that the urinary catheter has been changed 1304 or an override button 1306. The model may be further updated based on the interactions with the alert. Alarm 1308 is generated, for example, when the interaction journey of the current healthcare provider and the patient parameters are not correlated with other computed correlations of interaction journeys of other providers and patient parameters of other patients learned by the model. For example, the alert “patient with G6PD deficiency prescribed optalgin. Consult physician before dispensing” is presented when the model has learned that optalgin is not prescribed for patients with G6PD deficiency (or has no such learned correlation) and the current user has performed the correlation of interaction journey to prescribe optalgin to a patient with G6PD deficiency. A button 1310 may be pressed when the user addressed this issue.

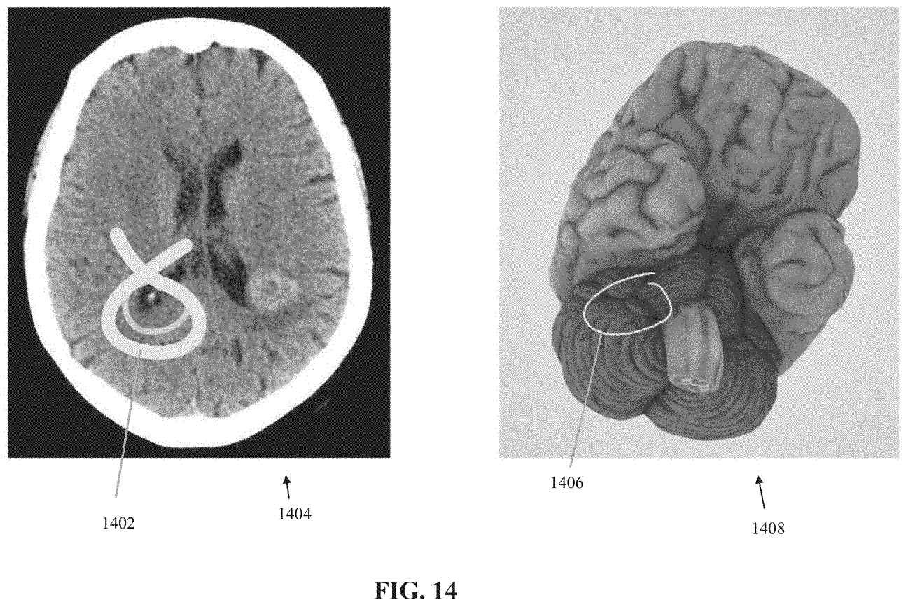

Reference is now made to FIG. 14, which is a schematic of an automated correlation between a marking 1402 on a 2D image 1404 (e.g., CT brain slice) and a corresponding computed marked location 1406 on a 3D image 1408 (e.g., 3D model or image of the brain), in accordance with some embodiments of the present invention. The automated correlation may be computed based on instructions outputted by the model, which is fed the 2D image 1402 and the interaction journey of the healthcare provider that made marking 1402 (e.g., by touching the image on the screen, using a digital pen to mark the location on the image on the screen, using a cursor on the screen controlled by mouse movement) and interaction journeys of other providers that presented a 2D image and beside it a 3D image and marked correlated regions on the images. Optionally, the 3D model may be automatically presented and correlated when the identity of the user is indicative that the user is a medical student, and/or other training stage, and/or is a teacher teaching students. Optionally, the 3D model may be automatically presented and correlated when the interaction journey indicates that the user is presently interacting with other healthcare providers. In this manner, the 3D model may be presented as a teaching aid and/or discussion aid.

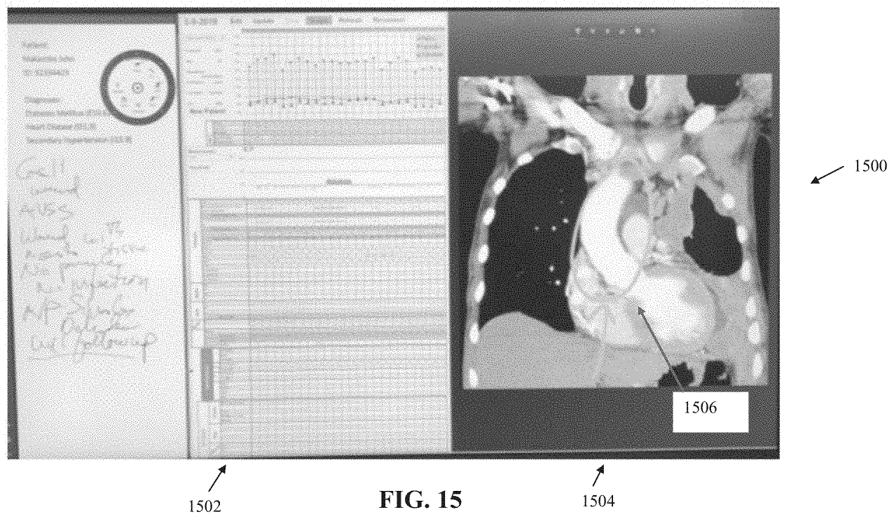

Reference is now made to FIG. 15, which is a schematic of a UI 1500 presented on a screen that is split into a data window 1502 for presenting patient parameters and an image window 1504 for presenting images, where the image is marked with a marking 1506 by a user, in accordance with some embodiments of the present invention. The image may be, for example, a 2D image, 3D image, static image, virtual reality (VR), augmented reality (AR), or multimedia item (inclusive, e.g., of video, movement, movie, sound, etc.), for example, as shown in FIG. 14. The interaction journey fed into the model (and/or which is learned by the model) may include one or more of the following: which data the user selected to present in window 1502 of the UI, the act of splitting the screen, which images the user selected to present in the window, and/or the user manually marking the screen.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant user interfaces, medical devices, patient parameters, interaction journeys and models will be developed and the scope of the terms user interfaces, medical devices, patient parameters, interaction journeys and models are intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of adapting a user interface (UI) for presenting medical data of a target patient, comprising:
monitoring an interaction journey of a healthcare provider with at least one medical device that performs at least one member of the group consisting of: storing data of a target patient, monitoring the target patient, presenting medical data of the target patient, and treating the target patient;
monitoring at least one patient parameter of the target patient;
feeding the interaction journey and the at least one patient parameter into a model trained according to at least computed correlation between interaction journeys of at least one of sample healthcare providers with respective medical devices and at least one patient parameter of at least one sample patient; and
outputting an adaptation to the UI by the model;
wherein the interaction journey is of a healthcare provider with a UI;
wherein the interaction journey is based on at least one member of the group consisting of: touch interaction with a touch screen presenting the UI, pen capable of writing on the touch screen, dial or wheel placed on a screen presenting the UI, a keyboard, a port receiving data from another user interface device or a network, a microphone, a virtual reality (VR) device, an augmented reality (AR) device, and/or a camera capturing images of the interaction with the UI.

2. The method of claim 1, wherein the at least one medical device is selected from the group consisting of: electronic health information system, electronic medical record, medical imaging system, medical image, patient monitor, anesthesiology monitor, physiological sensor or monitor, intracranial pressure sensor, cerebral perfusion pressure sensor, arterial line, respiration device, blood pressure sensor, temperature sensor, and pulse oximeter.

3. The method of claim 2, wherein the interaction journey is computed based on at least one member of the group consisting of: analysis of images captured by a camera of the healthcare provider interacting with the respective medical device, analysis of interaction of the healthcare provider with a dedicated display of the respective medical device, analysis of interaction of the healthcare provider with a generic display of a computing device connected to the respective medical device, and analysis of interaction of the healthcare provider with buttons of the respective medical device, interface of the medical device outputting data indicative of the interaction, and a microphone capturing interactions of healthcare providers with each other, interactions of healthcare providers with patients, and/or dictated patient notes, notes of patients.

4. The method of claim 1, further comprising obtaining at least one of sound and images and data of an interaction of the healthcare provider with at least one of the patient and other healthcare providers, and feeding the at least one of sound and images and data of the interaction into the model, wherein the model is trained on at least one of sound and images and data of a plurality of interactions of other healthcare providers with other patients and/or with another set of healthcare providers.

5-6. (canceled)

7. The method of claim 1, further comprising creating an adapted UI according to the adaptation outputted by the model, and iterating the monitoring the interaction journey, the monitoring the at least one patient parameter, the feeding, the outputting and the adapting, wherein the monitoring comprises monitoring the interaction journey of the healthcare provider with the adapted UI presented on a display.

8-9. (canceled)

10. The method of claim 1, wherein an interaction included in the interaction journey is selected from the group consisting of, zoom-in on a certain monitored patient parameter, selection of a certain monitored patient parameter for presentation in the UI, removal of a certain monitored patient parameter from the UI, relative positioning between two or more monitored patient parameters in the UI, marking a certain monitored patient parameter with an indication of importance, entering data, entering a diagnosis, entering orders for treatment, receiving patient parameter from a medical device and/or system, observing a change in a patient parameter.

11. The method of claim 1, wherein the interaction journey is computed based on at least one member selected from the group consisting of a camera capturing images of the healthcare provider treating the patient, a camera capturing images of healthcare provider actions when not directly treating the patient, a camera capturing images of healthcare provider washing hands, a camera capturing images of patient events including cough, seizure, sneeze, and/or fall, a microphone recording sounds captured during the patient events, a microphone recording sound captured during activities taking place in proximity to the patient, a microphone recording sound captured of the healthcare provider, and interactions of the healthcare provider with an input device.

12. The method of claim 1, wherein the at least one patient parameter is indicative of a current medical state relative to a target medical outcome of the target patient, wherein the model is trained according to computed correlations with a current medical state relative to a target medical outcome associated with each of the at least one sample patient, and wherein the adaptation to the UI outputted by the model is computed for increasing likelihood of the current medical state reaching the target medical outcome.

13. (canceled)

14. The method of claim 12, wherein the target medical outcome for the target patient is determined by correlating the respective at least one patient parameter and interaction journey to aggregated medical data collected from a plurality of subjects, and extracting the target medical outcome from the aggregated data.

15. The method of claim 1, wherein the adaptation to the UI is selected from the group consisting of: zoom-in on a certain monitored patient parameter, marking a certain monitored patient parameter to attract attention of the healthcare provider, selection of a certain monitored patient parameter that is not presented in the UI for presentation in the UI, removal of a certain monitored patient parameter from the UI, relative positioning between two or more monitored patient parameters in the UI, presenting a message indicative of a recommendation for manual adaptation of the UI, presenting at least one aggregated medical data collected from a plurality of subjects, presenting suggested diagnoses in the UI, presenting a suggested treatment plan in the UI, and presenting one or more parameter of a customizable period of time in the UI, playing an audio message on speakers, presenting an augmented reality image on an augmented reality headset, and presenting a virtual reality image on virtual reality glasses.

16. The method of claim 1, further comprising: receiving an indication of an identity profile of the healthcare provider, feeding the identity profile of the healthcare provider into the model, wherein the model is trained on computed correlations according to identity profiles of the at least one sample healthcare providers.

17. The method of claim 16, wherein the identity profile includes one or more members selected from the group consisting of: position, nurse, medical student, resident, staff physician, medical training rank, medical training, external and/or internal reviews, medical publication citations, medical specialty, demographic data, previous experience in performing medical procedures, medical skills, and success in treating other patients.

18-19. (canceled)

20. The method of claim 1, wherein the at least one patient parameter includes output of a plurality of physiological sensors that each measure a respective physiological parameter of the patient; wherein at least one patient parameter outputted by the plurality of physiological sensors are selected from the group consisting of: medical images, blood pressure measurement devices, arterial line, respiration devices, resuscitation devices, monitors, body temperature measurement devices, patient monitors, intracranial pressure sensors, Cerebral perfusion pressure sensors.

21. (canceled)

22. The method of claim 1, wherein the at least one patient parameter is obtained from a plurality of non-physiological data sources that each store a respective non-physiological parameter of the patient; wherein the plurality of patient parameters obtained from the plurality of non-physiological data sources are selected from the group consisting of: patient demographics, identity profile of healthcare providing team members, history of the present illness, prior medical history, prior treatments, previously scheduled appointments, future scheduled appointments, treatment facilities where the target patient was treated.

23. The method of claim 1, wherein the interaction journey and the at least one patient parameter are distributed to each of a plurality of processing nodes each hosting a respective model trained according to a unique computed correlations between interaction journeys of a plurality of unique healthcare providers with respective medical devices and the plurality of patient parameters of a plurality of unique sample patients, wherein outputs of the respective models are aggregated into an aggregated model and/or a single output of an adaptation to the UI.

24. The method of claim 1, further comprising feeding at least one aggregated medical data into the model, wherein the model is trained according to computed correlations between aggregated medical data and the interaction journeys of the at least one sample healthcare provider.

25-26. (canceled)

27. The method of claim 1, wherein the at least one patient parameter is obtained from a plurality of care process data sources; wherein each of the plurality of care process data sources stores a respective care process parameter of the patient; wherein the plurality of care process data sources are selected from the group consisting of: which parameters each caregiver referred to, identity of treating caregiver, position of treating caregiver, expertise of treating caregiver, decisions of processes chosen by treating caregiver, actions of treating caregiver within the system, requests and referrals of treating caregiver; clicks, searches, time spent on each parameter, system overrides, corrections, re-checks, focuses of treating caregiver.

28-29. (canceled)

30. The method of claim 1, wherein the at least one medical device comprises a plurality of medical devices, and the interaction journey of the healthcare provider is with the plurality of medical devices.

31. (canceled)

32. A system for adapting a user interface (UI) for presenting medical data of a target patient, comprising:
at least one hardware processor executing a code for:
monitoring an interaction journey of a healthcare provider with at least one medical device that performs at least one member of the group consisting of: storing data of a target patient, monitoring the target patient, presenting medical data of the target patient, and treating the target patient;
monitoring at least one patient parameter of the target patient;
feeding the interaction journey and the at least one patient parameter into a model trained according to at least one computed correlation between interaction journeys of at least one of sample healthcare providers with respective medical devices and at least one patient parameter of at least one sample patient; and
outputting an adaptation to the UI by the model;
wherein the interaction journey is of a healthcare provider with a UI;
wherein the interaction journey is based on at least one member of the group consisting of: touch interaction with a touch screen presenting the UI, pen capable of writing on the touch screen, dial or wheel placed on a screen presenting the UI, a keyboard, a port receiving data from another user interface device or a network, a microphone, a virtual reality (VR) device, an augmented reality (AR) device, and/or a camera capturing images of the interaction with the UI.

33. A computer program product for adapting a user interface (UI) for presenting medical data of a target patient, comprising:
a non-transitory memory storing thereon code for execution by at least one hardware process, the code including instructions for:
monitoring an interaction journey of a healthcare provider with at least one medical device that performs at least one member of the group consisting of: storing data of a target patient, monitoring the target patient, presenting medical data of the target patient, and treating the target patient;
monitoring at least one patient parameter of the target patient;
feeding the interaction journey and the at least one patient parameter into a model trained according to at least one computed correlation between interaction journeys of at least one of sample healthcare providers with respective medical devices and at least one patient parameter of at least one sample patient; and
outputting an adaptation to the UI by the model;
wherein the interaction journey is of a healthcare provider with a UI;
wherein the interaction journey is based on at least one member of the group consisting of: touch interaction with a touch screen presenting the UI, pen capable of writing on the touch screen, dial or wheel placed on a screen presenting the UI, a keyboard, a port receiving data from another user interface device or a network, a microphone, a virtual reality (VR) device, an augmented reality (AR) device, and/or a camera capturing images of the interaction with the UI.
